SEQUENCE LISTING (13 sequences)

SEQ ID NO: 1 - protein, 8 aa, Artificial Sequence
  Description of Artificial Sequence Synthetic peptide
DYKDDDDK

SEQ ID NO: 2 - protein, 9 aa, Artificial Sequence
  Description of Artificial Sequence Synthetic peptide
YPYDVPDYA

SEQ ID NO: 3 - protein, 10 aa, Artificial Sequence
  Description of Artificial Sequence Synthetic peptide
EQKLISEEDL

SEQ ID NO: 4 - protein, 4 aa, Artificial Sequence
  Description of Artificial Sequence Synthetic peptide
  MOD_RES (3): Arg or Gly
VPXS

SEQ ID NO: 5 - protein, 4 aa, Artificial Sequence
  Description of Artificial Sequence Synthetic peptide
IEGR

SEQ ID NO: 6 - protein, 5 aa, Artificial Sequence
  Description of Artificial Sequence Synthetic peptide
DDDDK

SEQ ID NO: 7 - protein, 6 aa, Artificial Sequence
  Description of Artificial Sequence Synthetic peptide
  MOD_RES (6): Gln or Gly
ENLYFX

SEQ ID NO: 8 - protein, 7 aa, Artificial Sequence
  Description of Artificial Sequence Synthetic peptide
  MOD_RES (6): Gln or Gly
LEVLFXP

SEQ ID NO: 9 - DNA, 27 bp, Artificial Sequence
  Description of Artificial Sequence Synthetic primer
cccgaattcatgtcttttctacaaaat

SEQ ID NO: 10 - DNA, 28 bp, Artificial Sequence
  Description of Artificial Sequence Synthetic primer
cccactagtctacctatccctcaactcc

SEQ ID NO: 11 - DNA, 96 bp, Artificial Sequence
  CDS (1)..(48)
  Description of Artificial Sequence Synthetic oligonucleotide
gatctggttccgcgtggatccccgggaatttccggtggtggtggtggaattctagactccatgggtcgactcgagtaagcttggtaccgcggctag

SEQ ID NO: 12 - protein, 16 aa, Artificial Sequence
  Description of Artificial Sequence Synthetic peptide
DLVPRGSPGISGGGGG

SEQ ID NO: 13 - protein, 5 aa, Artificial Sequence
  Description of Artificial Sequence Synthetic peptide
AWYRG

(19) United States
(12) Patent Application Publication
(10) Pub. No.: US 2007/0243579 A1
(43) Pub. Date: Oct. 18, 2007

(54) Selection Markers Useful for Heterologous Protein Expression

(76) Inventors: Steven Sedgwick, London (GB); Marco Geymonat, London (GB)

Correspondence Address: ARENT FOX PLLC, 1050 CONNECTICUT AVENUE, N.W., SUITE 400, WASHINGTON, DC, 20036, US

(21) Appl. No.: 10/588,505

(22) PCT Filed: Feb. 4, 2005
(86) PCT No.: PCT/GB05/00372

§ 371 Date: Feb. 8, 2007

(30) Foreign Application Priority Data
Feb. 6, 2004 (GB) 0402660.5

(51) Int. Cl. C12N 15/00; C12N 15/74; C12N 5/06; C12P 1/04

(52) U.S. Cl. 435/69.1; 435/320.1; 435/325; 435/476

(57) ABSTRACT

Selection markers in prior art systems are based on resistance genes or on complementation of auxotrophic mutations. The requirement for expression of these markers is conditional e.g. on the presence of an antibiotic, or on the absence of a nutrient. In contrast, the selection markers used in this invention arc non-conditional, and selection pressure is-absolute. The markers involved are genes which encode essential survival factors, such that loss of the marker gene is lethal. Thus the invention provides a cell which expresses chromosomal genes and extra-chromosomal genes, wherein (a) the expressed extra-chromosomal genes include an essential gene whose expression is unconditionally required for survival of the cell, (b) the expressed chromosomal genes do not include said essential gene, and (c) the extra-chromosomal genes include a heterologous gene. The cells can conveniently be obtained by a plasmid shuffling procedure.

All documents cited herein are incorporated by reference in their entirety.

### TECHNICAL FIELD

This invention is in the field of the recombinant expression of proteins in heterologous hosts.

### BACKGROUND ART

Recombinant expression of proteins is of huge importance. For convenience, bacterial hosts such as E. coli are typically used. Where bacterial hosts are unsuitable (e.g. where protein glycosylation or other modifications are desired, or where proteins are not expressed for one reason or another) it is common to choose a yeast host, a baculovirus host, or perhaps a cell line derived from a higher eukaryote, such as a CHO cell line. Plants are also used as recombinant expression hosts.

Although recombinant protein expression is often routine, with off-the-shelf kits being available for general use, many proteins cannot easily be expressed in this way. Bacterial hosts often give insoluble proteins which must be purified and re-folded from inclusion bodies, and do not offer eukaryotic post translational modifications. Yeasts (including Saccharomyces) grow poorly when minimal media are required by the selection systems that are commonly used, and Pichia systems [1] are generally useful only for secreted proteins. The baculovirus and CHO systems are cumbersome and expensive, and do not store well by freezing. Plant systems are at an early stage and extensive post-expression processing is required. Moreover, transformed hosts are typically unstable such that it is constantly necessary to impose selective conditions to prevent reversion to a non-transformed state e.g. by loss of expression plasmids, etc. For these reasons, hosts such as Saccharomyces are seen as poor choices for general recombinant expression.

Thus there remains a need for an expression system which avoids the need for expensive reagents, which is genetically stable, which can be frozen well, which can grow quickly and abundantly, and which can produce eukaryotic proteins in a soluble and active form. It is an object of the invention to provide an improved expression system to address these needs.

### DISCLOSURE OF THE INVENTION

The invention is based on the use of a new class of selection marker in expression vectors.

Selection markers used in prior art systems are often based on including a resistance gene in the vector e.g. an antibiotic resistance gene (e.g. ampicillin resistance, ampR), a drug resistance gene (e.g. neomycin resistance), a herbicide resistance gene (e.g. glyphosate resistance), the HPRT/HAT system, etc. When used with a host that is naturally sensitive to the factor in question, the resistance genes mean that only transformed cells can survive in a medium containing the factor.

Other selection markers are based on auxotrophic hosts i.e. those which require a particular factor in order to survive. Auxotrophic host systems are by far the most commonly used for yeasts [2], usually using URA3 (for uracil auxotrophs), LEU2 (for leucine auxotrophs), TRP1 (for tryptophan auxotrophs) or HIS3 (for histidine auxotrophs) to complement the mutations in the auxotrophic host and confer prototrophy. The hosts can grow in rich medium, but growth in a medium lacking an essential factor (e.g. lacking leucine) leads to cell death. Inclusion of a survival gene (e.g. the 2-isopropyl malate dehydrogenase encoded by LEU2) on a plasmid ensures that growth in the appropriate minimal medium selects only transformants. On transfer to a rich medium, where selection pressure is absent, auxotrophic hosts tend to lose plasmids encoding the selection markers.

These prior art selection systems are based on using a growth medium in which only transformants can survive, either by including the lethal factor (transformants are resistant) or by omitting the essential factor (transformants are not auxotrophic). The markers are thus conditional, as the selection pressure applies only under certain conditions. In contrast, the selection markers used according to the present invention are non-conditional i.e. the selection pressure is absolute. The markers involved are genes which encode essential survival factors, and loss of the marker gene (e.g. by loss of the expression vector) is lethal. By avoiding resistance markers, lethal factors (e.g. antibiotics) do not have to be added to culture media, thus simplifying the culture process, reducing costs and avoiding contamination of the expressed protein. By avoiding auxotrophic hosts, cells can be grown in rich media rather than in minimal media, thereby giving much better growth rates.

Thus the invention provides a cell that expresses both chromosomal genes and extra-chromosomal genes, wherein (a) the expressed extra-chromosomal genes include a gene with an essential function, the expression of which is unconditionally required for survival of the cell, (b) the expressed chromosomal genes do not provide that essential function, and (c) the extra-chromosomal genes include a heterologous gene, the expression of which is controlled by a promoter that is functional in the cell. Loss of the extra-chromosomal essential gene is lethal to the cell.

The invention also provides a method for expressing a heterologous gene, comprising the step of growing a cell of the invention in a culture medium. The invention also provides a method for purifying a protein, comprising the steps of: (a) growing a cell of the invention such that it expresses said protein; and (b) purifying the protein. The method may involve the step of: (c) treating the protein with a protease to provide a cleavage product of interest, and this step (c) may follow step (b) or may be an intrinsic part of step (b).

The cell of the invention can be constructed in two steps, as illustrated for yeast in FIG. 6 and as described below. The invention uses a starting cell that expresses both chromosomal genes and extra-chromosomal genes, wherein (a) the expressed extra-chromosomal genes include a gene with an essential function, the expression of which is unconditionally required for survival of the cell, (b) the expressed chromosomal genes do not provide that essential function, and (c) the extra-chromosomal genes include a conditionally-lethal gene.

The invention also provides an intermediate cell which expresses chromosomal genes, a first set of extra-chromosomal genes and a second set of extra-chromosomal genes, wherein (a) the expressed first and second sets of extra-chromosomal genes both include a gene with the same essential function, the expression of which is unconditionally required for survival of the cell, (b) the expressed chromosomal genes do not provide that essential function, (c) the first set of extra-chromosomal genes includes a conditionally-lethal gene, and (d) the second set of extra-chromosomal genes includes both a conditionally-required gene and a heterologous gene.

The invention also provides an extra-chromosomal vector, comprising: (a) an essential gene whose expression is unconditionally required for survival of a cell of interest; (b) a conditionally-required gene to allow selection of host cells which include the extra-chromosomal vector; and (c) a gene encoding a heterologous protein of interest operably linked to a promoter that is functional in the cell of interest.

The invention also provides a method for preparing a cell of the invention, comprising the steps of: (a) obtaining a starting cell, which expresses a conditionally-lethal gene; (b) transforming the starting cell with an extra-chromosomal vector of the invention; (c) selecting transformants which express the vector's conditionally-required gene; and then (d) selecting transformants which lose the conditionally-lethal gene.

The invention alternatively provides a cell which expresses chromosomal genes and extra-chromosomal genes, wherein (a) the expressed extra-chromosomal genes include an essential gene whose expression is unconditionally required for survival of the cell, (b) the expressed chromosomal genes do not include said essential gene, and (c) the extra-chromosomal genes include a heterologous gene, the expression of which is controlled by a promoter that is functional in the cell.

Essential Genes

The invention is based on the use of genes with essential functions as selection markers. Vectors encoding heterologous products of interest also encode the essential gene. As loss of the essential function is unconditionally lethal, the selection pressure for cells which contain the vector is absolute i.e. surviving cells must contain the vector with both the essential gene and the heterologous gene.

The essential gene can be any gene whose loss prevents the growth of cells e.g. the loss prevents cell division, prevents mitosis, prevents transcription, prevents translation, or prevents any other metabolic process which is essential for survival in culture. A gene is not an “essential gene” if its expression is required for survival only under certain conditions e.g. ampR is essential in the presence of ampicillin, but it is not essential under other circumstances, and so ampR is not an “essential gene”—its loss is not unconditionally lethal, as a change in growth conditions cannot compensate for the loss of an “essential gene”.

The identification of essential genes is straightforward e.g. using knockout studies, etc. Reference 3 lists various essential genes in E. coli, including some which are only conditionally-lethal, and the profile of the E. coli chromosome in reference 4 classifies genes as non-essential or essential. Reference 5 lists various essential genes for yeast, and the EUROSCARF [6] and EUROFAN [7,8] projects have also identified essential genes in yeast. EUROFAN defines an essential gene as one which is “imperative for the vegetative life cycle of a yeast cell grown on rich YPD media at 30° C.”, and estimated that 16-18% of yeast genes were essential on the basis that “a strain deleted for such a gene cannot grow on YPD at 30° C.”. As well as these functional studies, genomics (particularly comparative genomics) is often used to identify essential genes [9], and has been applied to E. coli, yeasts, Mycobacterium tuberculosis [10], etc. A further approach to identifying essential genes is given in reference 11. The DEG “database of essential genes” [12,13] is a further source. The skilled person is thus readily able to identify various genes whose absence cannot be tolerated by a host.

The essential gene is preferably short e.g. with a coding sequence (start codon to stop codon inclusive) of ≦3000 base pairs (e.g. ≦2500 bp, ≦2000 bp, ≦1500 bp, ≦1250 bp, ≦1000 bp, or shorter). The use of short genes is preferred because it reduces the potential for duplication of restriction sites within a vector. If restriction sites are duplicated, however, then codons can be changed to remove the recognition sequence without changing the encoded amino acid(s) or, as an alternative, the vector may be equipped for ligase independent cloning (LIC) as described below.

One advantage of the invention is that high copy numbers of the heterologous gene can be obtained, and this is accompanied by hyper-expression of the essential gene. Thus the essential gene is preferably not lethal when hyper-expressed. To achieve maximum copy number, it is preferred that the essential gene should be required by the host at high levels.

Preferred essential genes include those which encode polypeptides with (a) a molecular weight of less than about 40 kDa (e.g. <30 kDa, <20 kDa, or <10 kDa), and/or (b) reasonable cellular abundance as indicated by their codon adaptation indices (CAI [14]) of more than about 0.3. Genes which satisfy these criteria in yeast include: CDC33, COF1, EFB1, ERG25, FBA1, GPIV1, GSP1, GUK1, HEM13, HSP10, IPP1, NHP2, NOP1, NOP10, NTF2, PFY1, PSA1, RLP24, RPB10, RPC10, RPL5, RPL10, RPL15A, RPL17A, RPL18A, RPL25, RPL28, RPL30, RPL32, RPL33A, RPL43A3A, RPP0, RPS2, RPS3, RPS5, RPS13, RPS15, RPS20, RPS31, SAR1, SEC14, SMT3, SNU13, SSS1, SUI2, TIF11, TP11, VRG4, and YRB1.

Preferred essential genes include those involved in cell cycle control and/or involved in mitosis.

A preferred essential gene for use with the invention is MOB1, whose expression is absolutely required for completion of mitosis and maintenance of ploidy in yeast [15]. The yeast gene is less than 750 bp in length, and hyper-expression of the encoded Mob1 protein is tolerated.

Another preferred essential gene for use with the invention is Cdc33 (also known as eIF4E), which recognises the 7-methylguanosine-containing cap of mRNA in the first step of mRNA recruitment for translation. The Cdc33 protein has 212 aa in yeast and is abundant as judged by direct assays and by its CAI index of 0.387. Furthermore, as CDC33 is a translation factor then increased expression levels caused by copy number amplification may have a beneficial effect on heterologous protein expression. Over-expression of CDC33 can cause slow growth but this effect can be overcome in a Δcln3 or Δcln2 background [16] and should not matter anyway over a typical 4-8 hr induction period.

Another preferred essential gene for use with the invention is Cdc28, which is a protein of 298 aa in yeast. It is a serine/threonine protein kinase which is essential for the completion of the start, the controlling event, in the cell cycle. More than 200 substrates have been identified. Another preferred essential gene for use with the invention is Hsp10, which is a 10 kDa mitochondrial chaperonin in yeast (homologue of E. coli GroES) that regulates the Hsp60 chaperonin (171. Hsp10 is involved in protein folding and sorting in mitochondria.

Other essential genes for use with the invention can be identified empirically e.g. by the use of chromosomal knockout techniques to identify lethal knockout mutations, combined with a test for whether the lethal effect can be reversed by supplying a copy of the knocked-out gene on a plasmid.

In cells of the invention, the essential gene is expressed from an extra-chromosomal element rather than from a chromosomal site. Loss of the extra-chromosomal gene results in death of the cell.

The use of an essential gene makes the system inherently stable and so is preferable to the use of a resistance gene for several reasons. For instance: the need for minimal selective media is avoided, thus giving higher growth rates; there is no risk of the final product being contaminated by the resistance molecule e.g. antibiotic contamination; and, for cells such as yeasts, the need for expensive anti-microbials is avoided.

As the invention utilises genes that are essential, the absence of that gene from a host's chromosome(s) means that a functional copy of the gene has been lost from the chromosome, to be replaced by the extra-chromosomal gene. It will be understood that the replacement gene need not be precisely the same as the gene which has been lost. Tolerable differences include point mutations that change the gene's sequence without changing the encoded amino acid sequence, point mutations that change the encoded amino acid sequence without functional consequence, the addition of fusion sequences (e.g. a GST fusion of MOB1 can be used to replace native MOB1), and the use of a gene that is different from the lost chromosomal copy (e.g. from a different species, or even a different type of organism) but which is functionally able to complement that loss. Taking S. cerevisiae as an example, therefore, the host could lack an essential gene which is complemented by the corresponding gene from S. pombe or from any other eukaryote. The use of a non-identical gene which is less efficient than the native chromosomal gene can further enhance copy number amplification, as described below. However, the use of extra-chromosomal genes which are the same as those found wild-type in the host organism s chromosome is not excluded.

Preparing the Cell

Cells of the invention have lost an essential gene on their chromosome(s), but complement that loss using an extra-chromosomal copy of the gene. As loss of an essential gene cannot be tolerated, it is not feasible to make cells of the invention simply by deleting the chromosomal copy and then transforming the mutant cells with a vector encoding the gene, because death means that there is no way of selecting for cells which lack the essential gene. Instead, cells of the invention can be prepared by means of “plasmid shuffling” [18], involving a transitional stage where cells possess the essential gene in two separate extra-chromosomal forms (e.g. see FIG. 6).

The overall shuffling process begins with a mutant cell that lacks a chromosomal copy of an essential gene, but which possesses a replacement copy on a first vector, which vector also contains a conditionally-lethal marker. A second vector of the invention (carrying (a) a further replacement essential gene, (b) a conditionally-essential marker, and (c) a heterologous gene) is then used, and transformants are selected on the basis of the vector's conditionally-selective marker. At this stage the cell contains two extra-chromosomal copies of the essential gene, one on a first vector which contains a negative selection marker and one on a second vector which contains a positive selection marker and a heterologous gene. Loss of either vector leads to retention of the essential gene, but only the second vector is useful for heterologous protein expression. Thus the process then proceeds to eliminate cells which retain the first vector, thereby selecting cells which possess only the second vector. This final selection uses the first vector's conditionally-lethal marker, to yield cells in which the essential gene and the heterologous gene are encoded by the same vector. The overall effect of this process, therefore, is to replace the first vector with the second vector. Cells which lose both vectors lose the essential gene and thus die.

The invention can be performed much more quickly than existing eukaryotic expression systems, such as Pichia and baculovirus, and essentially as quickly as with advanced bacterial expression systems. Once the desired DNA fragment is cloned into the plasmid of the invention, a yeast host expressing high levels of the protein can be prepared in less than two weeks.

Overall, the shuffling process involves: (a) a host cell with an inactive chromosomal essential gene, complemented by a ‘covering’ plasmid which supplies the essential gene and contains a counterselection marker; and (b) an expression plasmid which also supplies an essential gene and contains the heterologous gene of interest (usually under the control of a repressible promoter) plus a selection marker. The shuffling protocol swaps the two plasmids without going via a stage where the extra-chromosomal essential gene is lost.

In S. cerevisiae a covering plasmid will generally include the UR43 counterselection marker, the expression plasmid will include a selection marker (e.g. auxotrophic marker), and the expression of the heterologous product will be controlled by galactose repression of GAL1-10. The URA3 marker advantageously allows selection of starting cells which contain the covering plasmid and also, using FOA, allows counterselection of intermediate cells. Similar considerations apply in S. pombe, although the heterologous product may be controlled by thiamine repression of the nmt1 promoter.

In E. coli and other applicable bacteria a covering plasmid may include the sacB gene from B. subtilis. This gene prevents growth on sucrose, permitting counterselection. Unlike URA3 the sacB gene does not also allow a positive selection and so the covering plasmid will also include a marker such as kan^{R} for selecting suitable starting cells.

As an alternative to the sacB system, the rpsL system can be used. Cells carrying the wild type rpsL (Str^{sens}) are sensitive to streptomycin, but many rpsL mutations give streptomycin resistance (Str^{res}). If a cell has both Str^{sens} and Str^{res} genes, however, they remain sensitive to streptomycin. A covering plasmid can thus contain wild-type rpsL and kan^{R}. Using a Str^{res} starting cell and an expression plasmid with amp^{R} the intermediate cells can be selected based on ampicillin resistance. Loss of the covering plasmid can then be selected based on streptomycin resistance.

The combined use of the sacB and strA systems in E. coli is described in reference 19.

The invention uses a starting cell which expresses chromosomal genes and extra-chromosomal genes, wherein (a) the expressed extra-chromosomal genes include an essential gene whose expression is unconditionally required for survival of the cell. (b) the expressed chromosomal genes do not include said essential gene, and (c) the extra-chromosomal genes include a conditionally-lethal gene. Suitable starting cells have been described in the art for various essential genes [e.g. 20,21]. The invention provides a starting cell, characterised in that (i) the cell is a S. cerevisiae yeast, and (ii) the essential gene is MOB1, Cdc33 or Hsp10.

As an alternative to using a plasmid shuffling approach, it is possible to prepare cells of the invention from diploid cells that are hetero-allelic for an essential gene i.e. cells that contain a diploid genome but which express a functional form of the essential gene from only one haploid set of chromosomes.

The hetero-allelic cell is transformed with a plasmid encoding both the essential gene and the heterologous gene of interest and, after sporulation, haploids lacking a functional chromosomal gene are selected [22]. This technique is more complicated than plasmid shuffling, but may be preferred if there is frequent recombination between chromosomes and shuffling plasmids.

Extra-Chromosomal Genes and Vectors

Cells of the invention include extra-chromosomal genes, which are located on an extra-chromosomal vector. Such vectors do not include DNA of the mitochondria, chloroplasts or kinetoplasts (where applicable). Preferred vectors are capable of autonomous replication i.e. their copy number can exceed the copy number of the host cell's own chromosome(s). Preferred vectors are non-integrating (unlike the situation with prior art Pichia systems). The extra-chromosomal genes will generally be found on a plasmid or in a viral vector.

Plasmids of the invention include an essential gene, such that (a) the plasmid can complement the lack of that gene in a host's chromosome, and (b) loss of the plasmid is lethal to the cell.

Plasmids of the invention also include a heterologous gene.

Plasmids of the invention will usually also include a conditionally-required gene. This gene is not required for survival of a cell of the invention, but may be used during the cell's preparation (see below). Conditionally-required genes allow transformants to be selected under appropriate selective growth conditions, and may confer resistance to an otherwise-toxic substance (e.g. an antibiotic resistance gene, such as ampR, kanR, tetR, hyg, etc.; a drug resistance gene, such as aad, ble, dlzfr; hpt, nptII, aphII, gat, pac, neoR, etc.; a herbicide resistance gene, such as ban, pat, csr1-1, shpd, epsp, etc.; and other resistance genes, such as ble, bsd, gpt, hisD, trpB, hprt, tk) or treatment (e.g. irradiation, mutagenesis), or may complement an auxotrophic mutation in the host's chromosome (e.g. the URA3, LEU2, TRP1, HIS3, LYS2, ADE2, ADE3 genes; etc.). A preferred conditionally-required gene is TRP1, which can be used to select yeast transformants on the basis of growth in a Trp-free medium.

Other plasmids used in preparing host cells of the invention (e.g. plasmids used to prepare starting cells, and retained in intermediate cells of the invention) include the same essential gene as described above, but include a conditionally-lethal gene for counterselection. Cells containing these plasmids can thus be selectively killed. Typical conditionally-lethal genes encode proteins which convert non-toxic substances into toxic substances, and examples include, but are not limited to: URA3 (lethal in the presence of 5-fluororotic acid, FOA); LYS2 (lethal in the presence of α-aminoadipic acid as the primary nitrogen source); CAN1 (lethal in the presence of canavanine and absence of arginine); CYH2 (lethal in the presence of cycloheximide); Tk or thymidine kinase (lethal in the presence of ganciclovir or acyclovir); Cd or cytosine deaminase (lethal in the presence of 5-fluorocytosine); Ntr or nitroreductase (lethal in the presence of CB1954); sacB from B. subtilis (lethal in the presence of sucrose); rpsL and mutant rpsL (selection based on streptomycin sensitivity/resistance); etc.

Some conditionally-required genes (for “positive selection”) can also be used as conditionally-lethal genes (for “negative selection”), depending on growth conditions. For example, URA3 is a conditionally-required gene for uracil auxotrophs, but it is lethal when growth occurs in the presence of FOA. Similarly, thymidine kinase offers a salvage pathway in the presence of aminopterin, but is lethal in the presence of acyclovir. A further example, dao1 encoding D-amino acid oxidase (DAAO) has been described in plants [23], where selection is based on the differing toxicity of D-amino acids and their metabolites in plants, as D-alanine and D-serine are toxic to plants, but can be metabolised by DAAO to non-toxic products, while D-isoleucine and D-valine have low toxicity but are metabolised by DAAO into toxic keto acids. Where a process of the invention uses both a conditionally-required gene and a conditionally-lethal gene, however, different genes will usually be used.

As well as (a) the essential gene, (b) the conditionally-required gene, and (c) the optional heterologous gene, plasmids of the invention will typically include one or more of the following elements: (i) an origin of replication functional in a host cell of interest (e.g. functional in yeast, such as an ars1 element or, more preferably, a 2 μori element); (ii) a polylinker or multi-cloning site, containing a plurality (e.g. 2, 3, 4, 5, 6, 7, 8, 9, 10 or more) of restriction sites in the same or, preferably, in different reading frames e.g. see FIG. 4; (iii) a transcription termination sequence (e.g. T-ADH1, T-CYC1, etc.) and/or additional stop codons (TGA, TAA and/or TAG) downstream of one or more (preferably all) of the promoters and their coding sequences in the plasmid; and (iv) a stabilising sequence, such as stb. Transcription termination sequences can be included as part of a heterologous insertion rather than as part of a starting vector.

To function as a shuttle vector between eukaryotes and bacteria, thereby simplifying preparative work, the plasmid may also include one or more of: (v) an origin of replication functional in bacteria, such as the ColE1 origin of replication; and (vi) an antibiotic resistance marker suitable for selection of bacterial transformants. As an alternative to using bacteria for preparative work, gap repair cloning [24] can be used.

Where a vector is for bacterial expression and is used in a shuffling procedure, an intermediate cell of the invention will include both a covering plasmid and an expression plasmid. The origins of replication in these plasmids should be of different compatibility groups to ensure that they can occupy the same cell during shuffling (e.g. one ColE1-based plasmid and one P15A-based plasmid).

Heterologous Genes

Plasmids used in cells of the invention, and in intermediate cells, include a heterologous gene i.e. a gene not naturally expressed in the organism in which the plasmid is propagated. Transcription of the heterologous gene will generally be under the control of a promoter that is functional in the host cell, as expression of the gene cannot be achieved using a promoter that is inactive in the cell.

The heterologous gene preferably comprises a coding sequence from a eukaryote, more preferably from a higher eukaryote. For example, the heterologous gene may comprise an animal sequence e.g. from a mammal, such as a human sequence. As an alternative, the heterologous gene may comprise a coding sequence from a virus (preferably a eukaryotic virus), a parasite, a pathogenic bacterium, etc. Various types of heterologous genes can be used: (a) one type of heterologous gene is a sequence which encodes a polypeptide that is useful during protein purification, and to which a further sequence of interest may be fused to give fusion polypeptides; (b) a second type of heterologous gene is a sequence which encodes a fusion polypeptide, comprising a sequence useful during protein purification, fused to a further sequence of interest; (c) a third type of heterologous gene is a sequence of interest without any fusion sequence. Fusion expression (b) of a protein of interest is typical, but direct expression (c) is also useful. A gene sequence useful during protein expression (a) will not typically be expressed as a protein for its own sake but will be used as a starting material for preparing a fusion construct (b).

Polypeptides commonly used as fusion partners to assist in purification include, but are not limited to: glutathione-S-transferase (GST), purified using immobilised glutathione [25]; poly-histidine tags, purified by IMAC [26]; calmodulin-binding peptide (CBP), purified using immobilised calmodulin; maltose-binding protein (MBP), purified using immobilised amylose; a chitin-binding domain (CBD)., purified by binding to chitin; secretory signals; and the Flag epitope (DYKDDDDK) (SEQ ID NO: 1) [27], haemagglutinin epitope (YPYDVPDYA, HA-tag) (SEQ ID NO: 2), VSV-G epitope, thioredoxin or c-myc epitope (EQKLISEEDL) (SEQ ID NO: 3), purified by specific immunoaffinity chromatography. Thus a plasmid of the invention may include a sequence that encodes one of these polypeptides, optionally fused to a further sequence of interest. These two elements may be arranged in either order, N-terminus to C-terminus, but it is typical referred to have the further sequence downstream of (i.e. fused to the C-terminus of) the purification sequence.

The ability to express proteins as GST-fusions is an advantage over Pichia systems, as GST-fusions in Pichia typically fail to bind to immobilised glutathione. The ability to use poly-histidine tags is also an advantage over Pichia, where alcohol dehydrogenase protein co-purifies on IMAC columns. The invention avoids these difficulties.

Where the heterologous sequence is designed for fusing to further sequences, or where it is fused to a further sequence, it is typical to include a protease recognition sequence at the junction between the two (i.e. at or near the 3′ or 5′ end of the heterologous sequence). A protease can then be used to generate the protein of interest without its purification tag. The proteolytic cleavage can take place after purification of the fusion protein or, to simplify purification, can take place while the fusion protein is immobilised on an affinity column, allowing the cleaved protein of interest to elute while the purification tag remains immobilised. Protease recognition sites include, but are not limited to: VPR/GS (SEQ ID NO: 4) (Thrombin); IEGR (SEQ ID NO: 5) (Factor Xa Protease); DDDDK (SEQ ID NO: 6) (Enterokinase); ENLYFQ/G (SEQ ID NO: 7) (endopeptidase rTEV from tobacco etch virus); and LEVLFQ/GP (SEQ ID NO: 8) (human rhinovirus protease 3C). As an alternative to using a protease recognition sequence, a self-cleaving protein can be constructed based on inteins [28,29].

Prior to use with the invention, the heterologous gene will be prepared in a form suitable for insertion into a vector of the invention. This may be by digestion of nucleic acid containing the gene, using enzymes that are compatible with the insertion site in the vector of the invention, or by inclusion of addition of suitable sequences during preparation e.g. by PCR amplification.

The insert may be suitable for ligase independent cloning (‘LIC’ [30-32]). For example, the 5′ and 3′ regions of the insert may have long (e.g. ≧15 nucleotides) high level of sequence identity to the ends of the linearised vector (usually long sticky ends), thereby facilitating insertion of the sequence into the vector without needing ligase (or phosphatase).

The insert sequence may be directly from a natural gene, or may have been modified in some way e.g. to remove introns, to change codon usage, to introduce or remove restriction sites, etc.

The invention has been found to be particularly suitable for expression of proteins which have been difficult to express in existing systems. Lte1 (low temperature essential) [33] is a large yeast protein (>1400 amino acids) which cannot be expressed in E. coli, but using the invention is has been successfully expressed in soluble form as a GST-fusion (in both directions, N-terminus to C-terminus). Thus the heterologous gene may encode a protein with 300 or more amino acids (e.g. 350, 400, 450, 500, 600, 700, 800, 900, 1000 or more), although expression of proteins shorter than 300 amino acids (e.g. 200 or fewer amino acids) is not excluded. Yeast proteins Bfa1 and Bub2 are found naturally at low levels and were subject to considerable degradation in E. coli expression systems [34], but have now been expressed at high levels in soluble form as GST-fusions. Expression of yeast kinases CDC5, CDC15 and CDC28 in E. coli gives inactive proteins, but these three proteins have been expressed in active soluble form as GST-fusions in yeasts having chromosomal deletions of the proteins. Mammalian proteins such as Tpl2 have also been successfully expressed as GST-fusions. Some of these proteins have subsequently been prepared in pure form after thrombin cleavage to remove the GST moiety. Likewise, soluble SARS virus Nsp13 gene product, a putative mRNA Cap1 methyl transferase, has been expressed and cleaved from the GST affinity purification tag using human rhinovirus protease 3C.

Thus the heterologous gene is preferably expressed as a soluble protein, even in fusion form. The production of soluble proteins is an advantage when compared to bacterial expression systems.

Following expression according to the invention, proteins may adopt their native dimeric form in solution. Thus the heterologous gene may encode a protein which naturally forms an oligomer, such as a dimer, trimer, tetramer, pentamer, hexamer, etc.

For hetero-oligomeric proteins, it is possible to express multiple heterologous genes from the same plasmid, but it is preferred to use one plasmid per heterologous gene, in which case the invention generally uses one essential gene per monomer i.e. the chromosome of a host for expressing a hetero-dimer will have two inactive essential genes, with their functions being complemented by different plasmids. Stoichiometric expression can be achieved if the same promoter is used for each monomer, provided that the plasmids' copy numbers are the same.

The heterologous gene is generally different from the essential gene.

Control of Gene Expression

Plasmids for use with the invention include (a) an essential gene, and (b) a conditionally-required gene and/or a conditionally-lethal gene. For expression purposes, plasmids of the invention also include a heterologous gene. Expression of these genes is controlled by upstream promoters. Various promoters may be used, but the invention offers better expression if particular promoters are used.

The essential gene is preferably under the control of a repressible promoter. To increase expression levels, the invention exploits the background level of “leaky” expression driven by such promoters even when they are turned “off” e.g. by catabolite repression. As the essential gene is required for the host cell to survive, but the host cell does not have a copy of the essential gene on its own chromosome, there is a selective pressure to increase the plasmid's copy number. As the copy number increases, the overall expression of the essential gene increases such that the combined background expression is adequate for survival.

By repressing expression of the essential gene, therefore, the invention can achieve a high copy number of the plasmid. An increase in copy number also gives increased levels of the heterologous gene, thereby improving expression levels of the protein of interest. The process of the invention may thus include a step of increasing the copy number of a vector to at least 5 (e.g. to at least 10, 20, 30, 40, 50 or more). The use of “leaky” low level expression to increase copy number is known [35].

Copy number amplification can be further enhanced by using codons in the essential gene which are non-optimal for the host in question. Where further enhancement of this type is not required, however, the essential gene may be modified for optimum codon usage.

The heterologous gene is preferably under the control of a promoter that is both repressible and inducible. Rather than being used to increase copy number, however, this promoter is used to allow controlled expression of the protein of interest. When there is an increase in copy number of the plasmid, high levels of heterologous protein expression are achieved. It is thus useful to avoid expression of the heterologous gene until a desired time to avoid possible toxic effects of over-expression. For example, if Bfa1 or Clb6 is over-expressed then cells die. Thus the heterologous gene may encode a protein that is potentially toxic to the host during normal growth.

A typical repressible promoter system for use with the invention is based on the GAL1-1 promoters of Gal1 galactokinase I and Gal10 UDP-glucose 4 epimerase. These are tightly repressed by glucose but highly activated when galactose is the sole carbon source. In S. cerevisiae, the dual GAL1 and GAL10 promoters are juxtaposed in nature (within the P_{GAL1} element) and are transcribed in opposite directions, and this arrangement of promoters conveniently allows divergent repression of the essential gene (controlled by one of the pair, in one direction) and the heterologous gene (controlled by the other member of the pair, in the other direction) [36].

Other repressible promoters include, but are not limited to: the repressible acid phosphatase gene promoter (PHO5), which is activated at low inorganic phosphate levels [37,38]; the thiamine-repressible promoter (from nmt1), which is repressed by thiamine [39,40]; the metallothionein promoter (from MTT1), which is induced by Cd²⁺ [41] the copper transport protein promoter (from CTR3), which is repressed in the presence of copper ions [42]; a light-switchable system involving a

DNA-binding domain fused to phytochrome, a transcription activation domain fused to PIF3, grown in a medium containing phycocyanobilin, with red light being an activator and far-red light being a repressor [43]. In bacteria the IPTG-inducible lac promoter can be used.

The heterologous gene and the essential gene may be controlled by separate copies of the same promoter. Expression of the two genes is thus controlled together, although over-expression of the heterologous gene is not generally required for the invention to function.

To express heterologous proteins according to the invention, a promoter will be activated (e.g. by addition of an inducer, or by removal of a repressor). While the expressed extra-chromosomal genes in a cell of tile invention must include the essential gene, therefore, the heterologous gene may be expressed or non-expressed depending on prevailing circumstances.

Yeast engages its ubiquitination system to tag many proteins for degradation at the exit from G1 and in tile later stages of M phase. This tagging can interfere with the yield of some heterologous proteins in yeast, but can be prevented by arresting cells in early G1 or M phase. Cell cycle arrest can be achieved in various ways, including the use of a factor or of cell cycle inhibitors such as nocadazole. Expression methods of the invention may thus involve the use of such reagents.

During expression of the heterologous gene, a yeast may be in diploid or haploid form.

Host Cells

Because all organisms have essential genes, and the invention is based on the fundamental principle of moving an essential gene from the chromosome onto an extra-chromosomal element so that transformants can be selected, the invention is applicable to all organisms, including prokaryotes and eukaryotes. In particular, the availability of plasmid shuffling protocols for many organisms facilitates the widespread use of the invention. Because bacterial expression systems are already well-developed, however, the invention's benefits are most immediately useful in eukaryotes, including unicellular eukaryotes (such as yeasts) and multicellular eukaryotes (such as animals and plants). As the use of essential genes as markers avoids the need for antibiotics, however, the invention offers advantages over conventional systems in situations where even traces of antibiotics in the purified expression product cannot be tolerated.

The invention is particularly useful for yeasts. Yeast is an inexpensive organism to work with, can be stored easily by freezing, and has an extensive historical background in expression and genetic manipulation, and with the sequencing of the S. cerevisiae genome, genomics and proteomics of this organism have been heavily exploited. Many suitable clones and vectors for expression and selection are readily available, and these have been extensively studied and characterised. Furthermore, studies of the yeast proteome have shown that yeasts are extremely tolerant to the expression of genes in the form of fusion proteins, without loss of solubility or function [44,45].

Preferred yeasts are those which support plasmids and, for assisting in the preparation of cells of the invention, which exist in haploid and diploid forms. Budding yeasts are particularly preferred.

Yeasts include the following genera: Arthroascus, Arxiozyma, Bullera, Candida, Debaryomyces, Dekkera, Dipodascopsis, Endomyces, Eremothecium, Geotrichum, Hanseniaspora, Hansemula, Hormoascus, Issatchenkia, Kloeckera, Kluyveromyces, Lipomyces, Lodderomyces, Metschnikowia, Pachysolen, Pachytichospora, Pichia, Rhodosporidium, Rhodotorula, Saccharomyces, Saccharomycodes, Schizoblastosporion, Schizosaccharomyces, Schwaniomyces, Sporobolomyces, Sterigmatomyces, Sympodiomyces, Taphrina, Torula, Torulaspora, Torulopsis, Trichosporon, Yarrowia, Zygohansenula, and Zygosaccharomyces. Preferred genera for use with the invention are Saccharomyces, Schizosaccharomyces and Pichia. Common industrial yeast systems include Hansenula polymorpha, Kluyveromyces lactis, Yarrowia lipolytica, Saccharomyces carisbergensis, Saccharomyces ellipsoideus and Candida utilis, and particularly preferred species for use with the invention are Saccharomyces cerevisiae (budding or bakers yeast) and Schizosaccharomyces pombe (fission yeast [46]). Such yeasts are readily available to the skilled person.

Many E. coli strains optimised for recombinant protein expression are available e.g. BL21 and its derivatives.

The invention does not utilise wild-type cells as hosts, as the invention relies on the absence of an essential gene from the host's chromosome, with that absence being complemented by an extra-chromosomal copy of the gene. Thus the host's chromosome will be lacking a functional copy of an essential gene. Typically, therefore, the invention will use a host that has a knockout genotype for the essential gene in question. The knockout may remove or disrupt the whole or part of the chromosomal gene, in the regulatory region(s) and/or the coding region(s). Thus remnants of the essential gene may remain in the chromosome, but the overall effect will be that the host's chromosome cannot be transcribed and/or translated to produce the essential gene product in functional form. Knockout of essential genes is known in the prior art [e.g. 20,21] but complementation with extra-chromosomal copies of the genes has been used to study the essential gene itself rather than as a way of selecting for the presence of a different heterologous gene.

Knockout by homologous recombination is a preferred method for obtaining suitable host cells, and in particular knock-out by isogenic deletion. Replacement of a chromosomal gene with a marker gene is typical e.g. as a result of homologous recombination to insert an antibiotic resistance gene. Gene inactivation methods such as those disclosed in references 47 and 43 can easily be adapted by the inclusion of covering plasmids encoding an essential gene prior to the inactivation step. Other non-knockout methods of preventing expression of an essential protein include chromatin silencing, antisense and RNA silencing (e.g. RNAi) techniques, although such techniques are not preferred due to their reversible nature and to the difficulty in ensuring that vector-derived genes are not also inactivated. A further way of eliminating the chromosomal gene's function is by mutagenesis of codons encoding critical amino acids e.g. a single Arg-522-His mutation in the sigA gene encoding σ^{A} in Mycobacterium smegmatis is lethal, without the need for knockout of the whole coding sequence [49]. Thus the skilled person can readily generate a host cell in which a chosen essential gene has been disabled, either by preventing its expression (either at a transcriptional or translational level) or by allowing its expression but in an inactive form.

In addition to knockout of the essential gene, the host may include further mutations to remove undesirable phenotypes. These mutations may already be present in a starting yeast strain, or they may be introduced.

For example, many host cells express endogenous proteases which degrade heterologous proteins. but which are not essential to viability under laboratory conditions. Deletion of such proteases from the host improves recombinant protein expression. Thus a cell of the invention may include knockout mutations of one or more endogenous proteases. In yeast, deletion of PEP4 function (the saccharopepsin aspartyl protease [50]) is a preferred mutation. Other proteases which can be knocked out include Prb1, Prc1 and Cps1.

The host cell may have mutations in genes responsible to cell wall assembly, such that the cell wall is weakened in order to simplify post-expression processing of cells. Such mutations make cells more fragile, which may not be useful in a general laboratory bench setting, but would be very useful in a specific expression system at an industrial scale where simplification of downstream processing is a higher priority than benchtop resilience.

The host cell may have mutations to prevent slow growth e.g. deletion of cln73 or cln2 in yeast. A preferred strain is one which is able to produce a higher biomass than wild-type yeast under the same conditions. A mutant strain has been described which contains only a single hexose transporter, a hybrid of Hxt1 and Hxt7 [51]. This mutation restricts glucose influx and avoids overflow into lactate. This results in slow steady respiration of the glucose and a higher resultant biomass.

The host cell may also include heterologous genes encoding foreign proteins, such as those from non-native metabolic pathways. For example, heterologous glycosyltransferases and other glycosylation enzymes (e.g. mannosidases I and II, N-acetylglucosaminyl transferases I and II, uridine 5′-diphosphate (UDP)-N-acetylglucosamine transporter, etc.) may be expressed in order to increase the glycosylation repertoire of an expression host [52], and in particular to mimic human glycosylation. Native pathways may be inhibited or knocked out to assist in this approach [53].

Multiple Genes

The invention has been described above in terms of using a single essential gene as a marker. The invention can also be used with multiple essential genes as markers. Each gene with an essential function is (a) expressed extra-chromosomally, the expression of those genes being required for viability of the cell, wherein (b) the expressed chromosomal genes do not provide those essential functions. For example, preferred essential genes may include both MOB1 and CDC28. Therefore, the chromosomal genes may have both MOB1 and CDC28 knocked out, and the functions provided by these genes are instead provided by extra-chromosomal genes. In a further example, it is possible for more than two essential genes to be used as markers (e.g. the chromosomal genes may have the MOB1, CDC28 and Hsp10 genes knocked out). As mentioned above, a number of essential genes have been described and it is possible to knock out any number of these genes on the chromosome of the host cell. For each loss of an essential function from the chromosomal genes, that function must be replaced by proteins expressed from the extra-chromosomal genes, otherwise the cell cannot survive.

The extra-chromosomal genes that provide the essential function may be found on the same plasmid as each other, or on separate plasmids. Therefore if the expressed chromosomal genes lack three essential functions, then the extra-chromosomal genes may provide these essential functions using one, two or three different plasmids. Therefore a single plasmid may comprise one or more (e.g. 1, 2, 3, 4, 5, 6, 7, 8, 9, 10 or more) genes with essential functions.

If the chromosomal genes have ii essential genes knocked out, then there must be n extra-chromosomal essential genes. Each cell may comprise from 1 to n differerent plasmids, which together provide the function of the n different essential genes. Each of the plasmids is required by the cell for survival. If there are fewer than n plasmids, then at least one plasmid will comprise more than one essential gene. Loss of any of the essential extra-chromosomal genes is lethal to the cell.

The invention may also be used to express more than one heterologous protein, and the invention is then particularly useful for the co-expression of proteins that can interact to form complexes e.g. heterodimers. Each plasmid encoding an essential gene may also encode one or more (e.g. 2, 3, 4, 5, 6, 7, 8, 9, 10 or more) heterologous gene of interest.

The cell may express up to x heterologous proteins x can be the same as n, less than n or greater than n, depending on whether the essential gene and/or heterologous protein is duplicated.

Preferably, for n knocked out essential genes and n heterologous genes, the cell comprises n plasmids, each comprising one extra-chromosomal essential gene and one heterologous gene.

Therefore, the cell of the invention may comprise at least one further extra-chromosomal gene with an essential function that the chromosomal genes do not provide. The further extra-chromosomal genes may also comprise at least one further heterologous gene, the expression of which is controlled by a promoter that is functional in the cell. In such a case, loss of any of the extra-chromosomal essential genes is lethal to the cell.

Where more than one essential function marker is used, each is replaced by carrying out the plasmid shuffling steps described above, once for each particular plasmid encoding an essential gene. Each covering plasmid and each expression plasmid should contain a different conditionally lethal selection marker such that their loss can be selected individually.

For example, a cell may be a MOB1 and a CDC28 knock out. Such a cell may contain two covering plasmids; one which expresses MOB 1, the other expressing CDC28. In a first plasmid shuffling step the MOB1-encoding covering plasmid is replaced by a MOB1-encoding expression plasmid that also expresses at least one heterologous protein, and in a second plasmid shuffling step the CDC28 encoding covering plasmid is replaced by a CDC28 encoding expression plasmid that expresses at least one (different) heterologous protein.

Alternatively, the cell may contain a single covering plasmid which expresses both MOB1 and CDC28. Plasmid shuffling is then used to replace the single covering plasmid with the two expression plasmids, each of which expresses one or more (e.g. 1, 2, 3, 4, 5, 6, 7, 8, 9, 10 or more) heterologous genes. Cells are selected which contain the two expression plasmids.

It is also possible to replace a single covering plasmid which covers two knocked out essential genes with a single expression plasmid that comprises both essential genes and expresses one or more (e.g. 1, 2, 3, 4, 5, 6, 7, 8, 9, 10 or more) heterologous genes. It is also possible to replace two covering plasmids that comprise different essential genes with a single expression plasmid that covers both essential genes and expresses one or more (e.g. 1, 2, 3, 4, 5, 6, 7, 8, 9, 10 or more) heterologous genes.

It is also possible to carry out a similar process where more than two (e.g. 3, 4, 5, 6, 7, 8, 9, 10 or more) essential genes, more than two (e.g. 3, 4, 5, 6, 7, 8, 9, 10 or more) heterologous genes, more than two (e.g. 3, 4, 5, 6, 7, 8, 9, 1 O or more) covering plasmids and/or more than two (e.g. 3, 4, 5, 6, 7, 8, 9, 10 or more) expression plasmids are used.

General

The term “comprising” means “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

Polypeptides

The invention also provides polypeptides expressed by the methods of the invention. The polypeptides expressed by the invention may be expressed as single proteins or as complexes. For example, the polypeptides may be expressed as homo- or heterodimers. Preferably the polypeptides expressed using the invention are not expressable using conventional techniques known in the art. Preferred polypeptides are Lte1 protein, a Bfa1 protein, a Bub2 protein, a CDC5 protein, a CDC14 protein, a CDC15 protein (both wild type and kinase dead), a CDC16 protein, a CDC23 protein, a CDC28 protein, a Tpl2 protein, a SARS virus Nsp13 protein, a mRNA Cap2 methyl transferase protein, Cla4 protein, Dbf2 protein, APC1 protein, the PP2A subunits Tpd1, Pph21, Pph22, Cdc55 and Rts1, a Clb6 protein, an Rgd1 protein, a Ubc4 protein, a Plo1 protein, a HBP1 protein, a PLK1 kinase protein, a KIF2C protein, a CHO kinesin MCAK protein, a p105 protein, a human Abin2 protein, Mob1/Dbf2 N305A dimer, Mob1/Dbf2 dimer and TPL2/p105 dimer.

### BRIEF DESCRIPTION OF DRAWINGS

FIG. 1 illustrates the construction of starting strains for use with the invention, and

FIG. 2 shows a further development of this process, starting with the strain produced at the end of FIG. 1.

FIG. 3 shows two maps of the pMG1 plasmid, with FIG. 4 showing its polylinker site (SEQ ID NO: 11 and SEQ ID NO: 12).

FIG. 5 shows expression from the pMG1 plasmid using glucose (5A) or galactose (5B).

FIG. 6 shows the plasmid shuffling used in selecting cells of the invention. The yeast cell is shown progressing from starting cell to intermediate cell to a cell useful for heterologous expression of proteins according to the invention.

FIGS. 7 to 10 show the results of protein expression according to the invention. The lanes were loaded with protein from -30mi of culture.

FIG. 11 shows the MOB/TRP1-based vectors (A) pMH919 and (B) pGSTMob/Dbf2.

FIG. 12 shows a comparison of the yields of GSTI-Ubc4 when expression is induced with varying concentrations of galactose.

FIG. 13 shows the optimum glucose concentration for expression of GST-Tpl2.

FIG. 14 shows the purification of components of the S. cerevisiae mitotic exit network.

FIG. 15 shows (A) purification of GST-Cla4, 6His-Lte1 and GST-Lte1, (B) phosphorylation of 6His-Lte1 by GST-Cla4 and (C) guanine nucleotide exchange activity of Lte1 (x-axis shows time in minutes, y-axis shows % Tem1-GDP, diamonds are Bfa1+Tem1, squares are Bfa1+Tem1+Lte1).

FIG. 16 shows (A) the elution of GST-Cdc15, (B) the phosphorylation of Mob1/Dbf2 by Cdc15 and (C) the activation of Mob1/Dbf2 kinase by Cdc15.

FIG. 17 shows the purification and activities of GST-Mob1, wild type, kinase (lead and hyperactive Dbf2.

FIG. 18 shows the purification of S. cerevisiae APC components.

FIG. 19 shows the specific phosphorylation of GST-Cdc16 and GST-Apc1 by Dbf2/GST-Mob1.

FIG. 20 shows the purification of GST-Cdc14 and phosphorylation by Dbf2/Mob1.

FIG. 21 shows the phosphatase activity of GST-Cdc14 (y-axis is activity, x-axis is time) Activity is measured using absorbance at 410 nm.

FIG. 22 shows the phosphatase activity of wild type and mutant GST-Cdc14. Lane Key 1: wild type, 2:1-462, 3:1-372: 4:316-551, 5:462-551, 6: GST only, 7: S464A S467A and 3: S494A S496A S497A S498A.

FIG. 23 shows (A) the purification of GST-Net1 and (B) the inhibition of Cdc14 activity by Net1 (x-axis shows time in minutes, y-axis shows phosphatase activity [OD410 nm], diamonds are GST-Cdc14, squares are GST-Cdc14+GST-Ne1).

FIG. 24 shows the purification of the five subunits of S. cerevisiae protein phosphatase 2A.

FIG. 25 shows the phosphatase activity of PPH2A (y-axis is activity, x-axis is time) Activity is measured using absorbance at 410 nm.

FIG. 26 shows the purification of GST-Clb6 cyclin box fragments.

FIG. 27 shows the purification of GST-Rgd1.

FIG. 28 shows the large scale preparation of GST-Ubc4. Key: B-beads before elution, R-beads after elution.

FIG. 29 shows the phosphorylation of MIBP by S. pombe GST-Plo1.

FIG. 30 shows (A) the purification of mouse GST-Hbp1 and (B) the purification of SARS virus GST-Nsp13 methyltransferase.

FIG. 31 shows the purification of three GST-polo domain fragments from human polo-like kinase.

FIG. 32 shows the purification of the kinesins KIF2C and MCAK.

FIG. 33 shows (A) the expression of rat GST-Tpl2 and N- and C-terminal deletion derivatives, (B) human 6His-p105 and (C) human GST-Abin2

FIG. 34 shows the elution of GST-Tpl2.

FIG. 35 shows the interation of GST-Tpl2 and 6His-p105.

FIG. 36 shows vector maps of (A) pMH925 and (B) pMH927.

FIG. 37 shows the coexpression and copurification of GST-Tpl2 and 6His-p105.

### MODES FOR CARRYING OUT THE INVENTION

### Construction of Starting Yeast Strains

Diploid S. cerevisiae strains that are heterozygous for MOB1 (MOB1/mob1::kan^{R}) are available. Such a strain was obtained and was transformed with a pURA3 plasmid (“pRS316” [54]) carrying a BamHI-EcoRI PCR fragment encompassing the entire MOB1 coding sequence plus flanking regulatory elements [15]. This strain is gal2 has sub-optimal growth on galactose as a sole carbon source) and is Ura (requires uracil in growth medium). Ura⁺ transformants were selected and allowed to sporulate. After germination, haploid mob1::kan^{R} strains were selected using G418. These cells have lost their chromosomal MOB1, but its activity is complemented by the MOB1⁺ plasmid. These cells were mated with a second haploid strain (“CG379” [55]) which was MOB1 trp1 GAL2 and the mated diploid cells were then sporulated. Spores which were trp1 GAL2 nob1::kan^{R} (cannot grow without tryptophan, can grow on galactose, G418 resistant) were selected for G418 resistance and growth on galactose medium. One which was mating type a was designated MGY66 and had the following relevant genotype MATa mob1::kan^{R} trp1 GAL ura3 pURA3-MOB1. MGY66 is a suitable starting cell for use with the invention, and its overall construction is shown in FIG. 1.

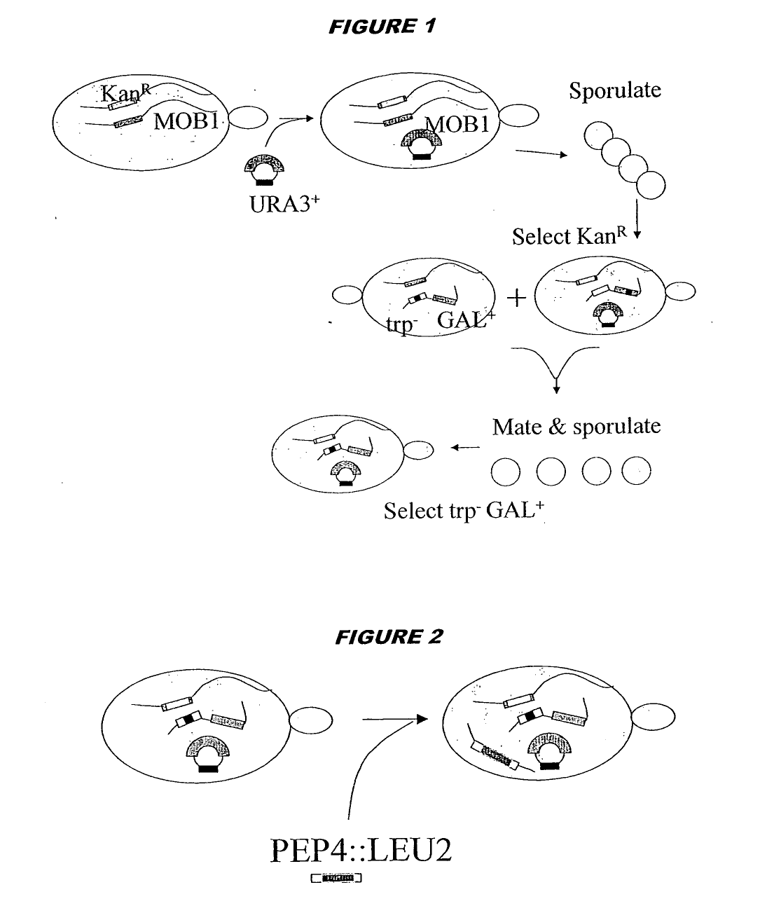

As a further development, shown in FIG. 2, the PEP4 gene of this strain was knocked out and replaced with a LEU2 cassette [56]. The resulting strain is referred to as “MGY70” and is MATa mob1::kan^{R} trp1 GAL pep4:.LEU2 ura3-pURA3-MOB1. The PEP4 gene encodes an aspartyl protease (“saccharopepsin”) which can degrade recombinantly-expressed proteins, but which is not essential for cell survival, and so its deletion can improve yields of stable recombinant proteins.

Preparation of Expression Plasmids

Starting with plasmid pESC-URA (Invitrogen™), a Pvu1 fragment was excised, which contains the divergent, conditional and galactose-inducible yeast Gal1-10 promoters and yeast ADH and CYC1 terminators. This fragment was used to replace a Pvu1 fragment of pRS424 [57] to give “pESC-424”.

An EcoRI-SpeI fragment encompassing the MOB1 coding sequence was made by PCR of yeast genomic DNA using the following primers:

(SEQ ID NO: 9)

| | |
|---|---|
| Fwd, with EcoRI site: | CCCGAATTCATGTCTTTTCTACAAAAT |

(SEQ ID NO: 10)

| | |
|---|---|
| Rev, with SpeI site: | CCCACTAGTCTACCTATCCCTCAACTCC |

The PCR fragment was cloned into the GAL10 promoter of pESC-424 to give pESC-424-MOB1. The same EcoRI site was then removed by infilling with Klenow DNA polymerase, to give “pESC-424-MOB1-ΔEcoRI”. Removal of this EcoRI site allowed a unique EcoRI site to be later included in a polylinker.

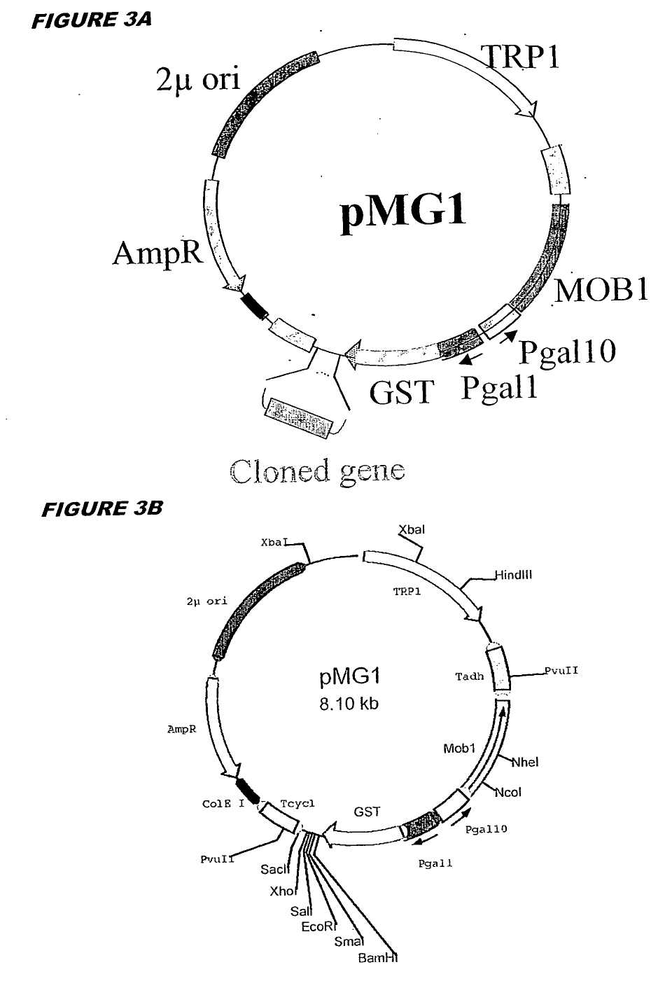

A BglI-XhoI fragment containing a GST coding sequence, a thrombin cleavage site and a polylinker was made by PCR of pGEX-KG [58] and cloned between BamHI and XhoI sites of pESC-424-MOB1-ΔEcoRI, to give the plasmid “pMG1” (FIGS. 3A & 3B). The polylinker site (FIG. 4) can receive genes encoding proteins of interest for expression as GST-fusions.

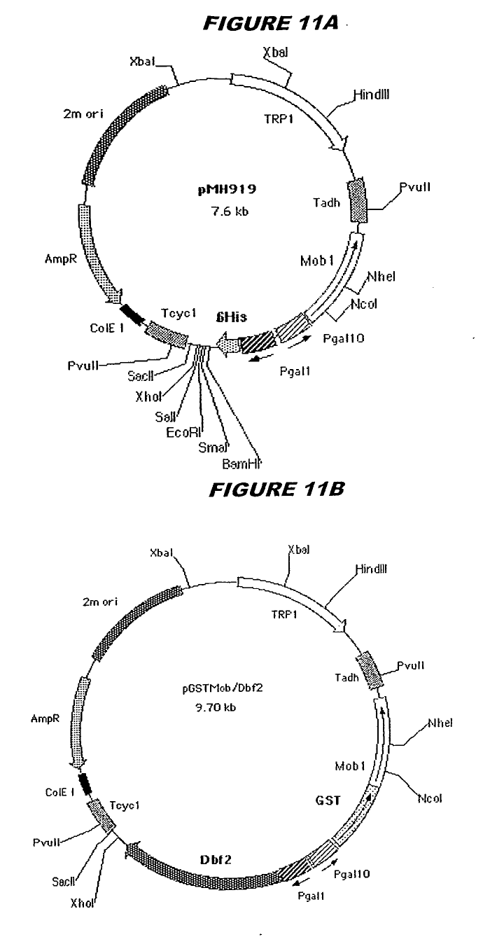

The plasmid pMH919 (FIG. 11A) was prepared using similar methods known in the art. The polylinker site of pMH919 can receive genes encoding proteins of interest for expression as 6His-fusions.

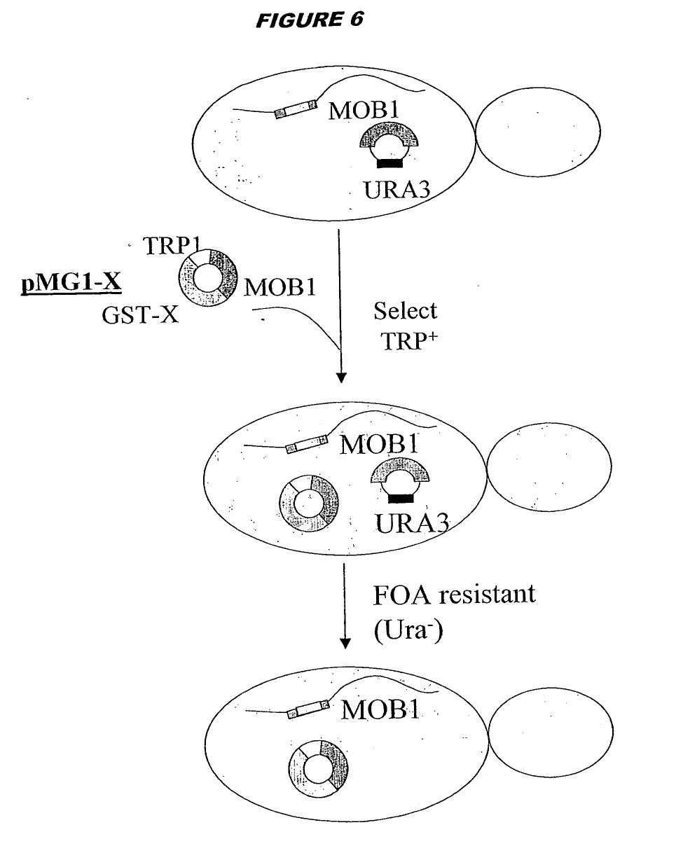

Transformational to Express Recombinant Proteins (FIG. 6)

Plasmid pMG1 is grown in E. coli and a plasmid DNA miniprep is prepared. Separately, a gene encoding a heterologous protein of interest is prepared which, after restriction enzyme treatment, will have sticky ends that are compatible and in-frame with the polylinker site in pMG1. The two molecules are digested and ligated to give a plasmid encoding the protein of interest in the form of a GST-fusion protein. This plasmid (“pMG1-X”) is transferred into MGY70 yeast by the lithium acetate protocol, and is then selected on a minimal medium lacking tryptophan. As MGY70 is trp1, only transformants survive. Next, the cells are grown on agar with uracil and 1 mg/ml 5-fluororotic acid, which selects against URA3⁺ cells. Surviving cells are those which have lost the pURA3-MOB1 plasmid, but which have retained pMG1-X as the sole source of MOB1.

The final transformants can be grown in rich media (e.g. in YEP medium) without further selection. The cells require uracil to grow, but this is supplied by rich media. The cells can be frozen at this stage to provide long-term stocks e.g. freezing at −80° C. in YEP medium with 20% glycerol.

Expression of the heterologous fusion protein can be induced by switching on the pGAL promoters.

Protein Expression and Purification

Yeast cells of the invention contains a heterologous gene under the control of a pGAL promoter. The MOB1 is also under the control of a pGAL promoter. This arrangement allows a very high copy number of the pMG plasmid to be achieved prior to expression of the heterologous gene, thereby giving high expression levels. Furthermore, by keeping the heterologous gene in an “off” state at this stage then any possible toxic effects of the heterologous gene are avoided.

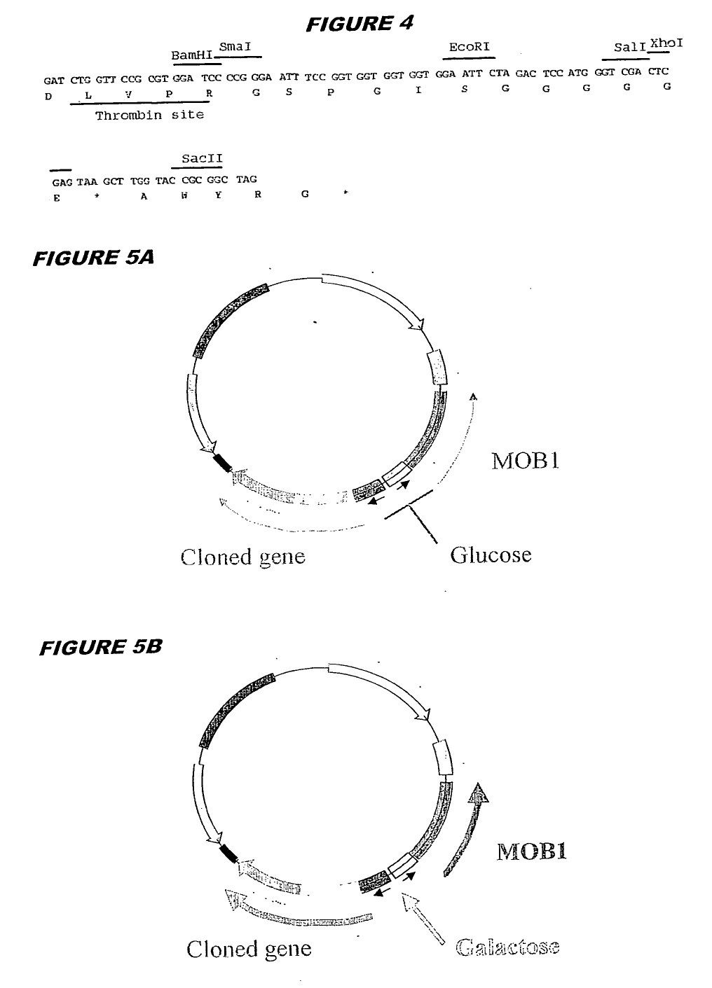

Cells need MOB1 expression to survive. As the MOB1 gene is under the control of a pGAL promoter, which is repressed when cells are grown on glucose, it would seem on paper that the cells would die when grown on glucose. As repression is not 100% efficient, however, there is a low-level basal expression from the pGAL promoters (FIG. 5A). This basal expression provides low levels of MOB1 to the growing cells, allowing survival. Moreover, the absolute need for MOB1 operates as a selection pressure to increase the copy number of pMG1. In the presence of glucose, therefore, the copy number of pMG1 increases to high levels.

When expression of the heterologous protein is desired, the cells are transferred to a galactose medium. The absence of glucose and presence of galactose removes repression of the pGAL promoters and expression of the heterologous protein is thus induced (FIG. 5B). Furthermore, the recombinant gene is expressed at even higher levels because of the high copy number resulting from the pGAL-controlled MOB1 selection.

After induction, cells are grown and then harvested. The cell lysate is applied to a glutathione column, which retains the GST-fusion protein. After washing, thrombin is added to the column. leading to elution of the cleaved heterologous protein in pure form.

Expression of Murine TPL2

This transformation/expression/purification process was followed for murine TPL2 protein.

A pCDNA3 vector carrying the cDNA of the complete mouse TPL2 coding sequence was used as a PCR template to generate a DNA fragment suitable for cloning into pMGY1. The PCR forwards primer-included the first 18 coding bases of TPL2 preceded by a synthetic BamHI site. The BamIHI site was designed to so that the TPL2 sequence was in frame with the 3′ end of the GST sequence of pMG1. The reverse primer had the last 18 bases of the negative strand in reverse 5′-3′ orientation preceded by a synthetic XhoI site. The PCR product was prepared for digestion using the Wizard PCR Preps DNA Purification System. The PCR fragment and pMG1 were digested with BamHI and XhoI restriction enzymes. The PCR fragment was again purified using the Wizard PCR Preps DNA Purification System. The digested vector was electrophoresed through a 10% agarose TAE buffered gel. Linear plasmid was excised from the gel and purified from the agarose using a Geneclean Kit. Vector and PCR fragments were ligated together by incubation together for 2 h. Control ligations were done with no insert DNA.

Ligation mixtures were transformed into E. coli DH10b. Transformed E. coli were selected on L agar containing 20 μg/ml ampicillin+20 μg/ml nafcillin. Individual clones were colony purified by restreaking on amp+naf selective medium. Miniprep DNA of individual clones was prepared using the Wizard Plus Minipreps DNA Purification System. Miniprep DNA was digested with BamHI+XhoI restriction enzymes to identify clones carrying the ˜1.6 kb TPL2 coding sequence.

The DNA of three potentially positive pMG1-TPL2 clones were transformed into S. cerevisiae MGY70 using the lithium acetate procedure. MGY70 transformants with this TRP1 plasmid were selected by growth at 30° C. on minimal agar medium lacking tryptophan. Two individual transformant clones obtained from each miniprep DNA sample were colony purified by re-streaking on agar medium lacking tryptophan. A single colony from each of these plates was streaked onto minimal medium supplemented with 20 μg/ml uracil and 1 mg/ml FOA. FOA plates were incubated for 2-3 days at 30° C. Single colonies were picked onto fresh FOA plates and grown for a further 2-3 days. In these cells the covering plasmid in MGY70 that provided the essential MOB1 gene had been replaced by the expression plasmid and its copy of MOB1. From this point onwards these cells could be grown on rich medium with no further conditional selection.

Examples of the resulting single colonies were next tested for protein expression. However, at this stage it was useful to test whether expression of the cloned gene in toxic as this influences the induction regime for inducible gene expression. Induction of toxic gene products is indicated by failure of the cells to grow on rich agar medium with 2% galactose as carbon source. Induction of the potential TPL2 clones was not toxic as judged by this simple test.

Three potential isolates originating from three independent ligation events were tested for expression of TPL2. 50 ml overnight cultures were grown at 30° C. in rich, YEP, medium with 2% raffinose as carbon source. The cultures were inoculated so that cell density after overnight growth was approximately 5×10⁷/ml. The overnight cultures were used to inoculate 500 ml of YEP medium supplemented with 2% galactose as carbon source and grown for 6-8 h at 30° C. Cells from 50 ml and 450 ml of culture were harvested by centrifugation, frozen rapidly on1 dry ice and stored at −80° C. The small pellets were used to check for induced expression of TPL2 while the larger pellets were held in reserve for preparation of Tpl2 for experimental use.

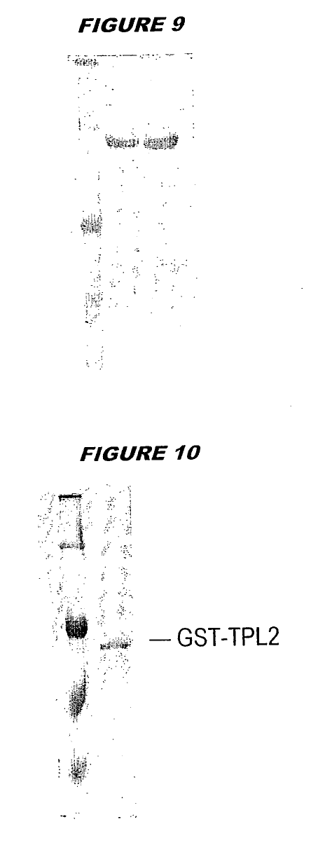

Small pellets were resuspended in 400 μl of lysis buffer (50 mM Tris-HCl pH 7.5, 250 mM NaCl, 1% Nonidet P40, 10% glycerol, 4 mM dithiothreitol, 200 μg/ml sodium orthovanadate, 10 mM NaF, 50 mM glycerol-2-phosphate, 1 mM PMSF, ‘Complete’ protease inhibitor (Roche™)). For cell lysis, glass beads, 0.5 mm diameter, were added to tile meniscus in 2 ml screw cap tubes which were then shaken three times 10 sec in a RiboLyser apparatus (Hybaid™). Cell lysate was recovered by piecing the base of the tube and followed by centrifugation inside a larger tube. Cell debris and insoluble material was removed by 2×15 min centrifugation at 13000 rpm in a refrigerated micro centrifuge. The cleared lysate was added to 50 μl of glutathione sepharose beads which had been pre-equilibrated in 250 mM NaCl, 50 mM Tris-HCl pH 7.5, 0.2% Nonidet P40. The beads were gently mixed with the lysate on a rotor at 4° C. for 1-2 h. The beads were washed 5× with 250 mM NaCl, 50 mM Tris-HCl pH 7.5, 0.2% Nonidet P40, 4 mM dithiothreitol. Proteins bound to the glutathione sepharose beads were analysed by SDS-polyacylamide gel electrophoresis. Protein bands were visualised by staining with coomassie blue (FIG. 10).

Large cell pellets were resuspended in lysis buffer (approximately 10 ml/1 g cells). Cells were lysed with a French pressure cell operating at 20000 psi. Cleared lysates were made by centrifugation at 18000 g for 2×20 min at 4° C. Large scale affinity purification of GST-TPL2 was essentially as described above except that appropriately increased amounts of reagents were used.

In contrast to the successful expression of TPL2 using the system of the invention, attempts to express the protein in ES-col using the pGEX-4t and pET28 plasmids failed. The attempts used the full length protein as well as deletion derivatives lacking the N-terminal 30 residues and/or the C-terminal 70 residues (an oncogenic form). The kinase domain on its own was also tested. In all cases, however, any product which was seen (very little) was heavily degraded, inactive, insoluble or aggregated and was thus of limited use.

Expression was also attempted without success using the Invitrogen™ DES system using the pMT/V5-His vector and S2 Drosophila cells.

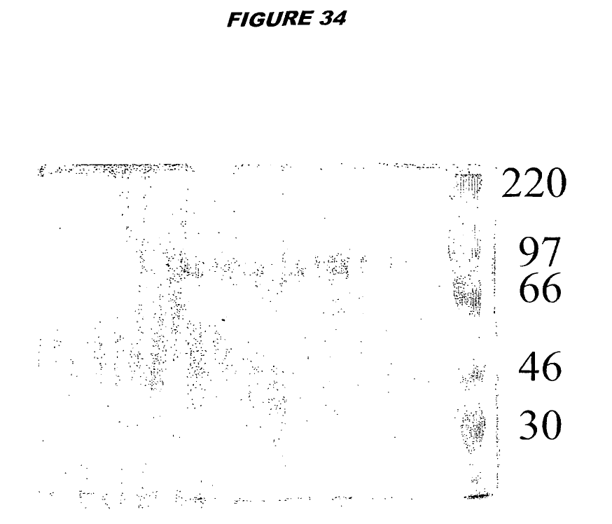

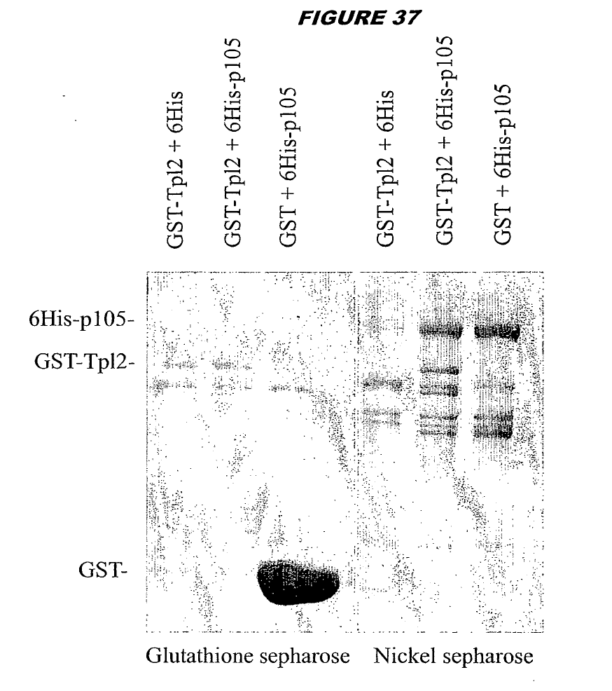

GST-Tpl2 from rat has also been expressed from a plasmid where CDC28 was used rather than MOB1 as the essential gene (See FIG. 37 and section regarding expression of two proteins below). Larger scale preparations of GST-Tpl2 yielded approximately 0.5 mg of protein from 25 g of induced cells (FIG. 34).

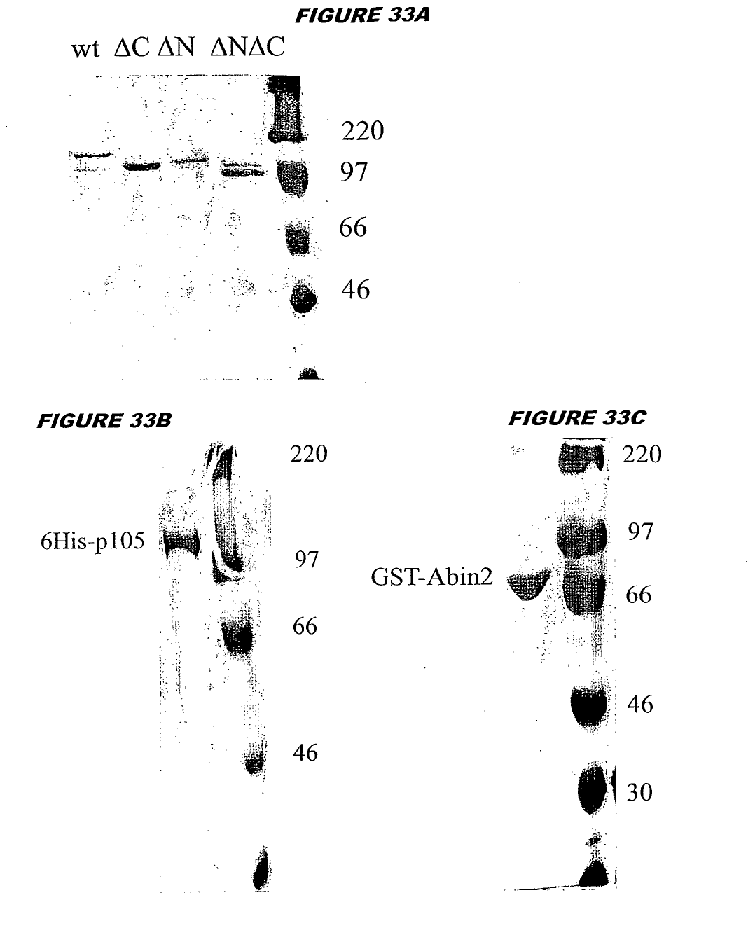

In addition to full length Tpl2, three deletion derivatives have also been expressed. An N-terminal deletion which lacks 30 residues, a C-terminal deletion lacking 78 residues which mimics a naturally occurring oncogenic form of the protein, and an N- and C-terminal derivative combines both of these deletions (FIG. 33).

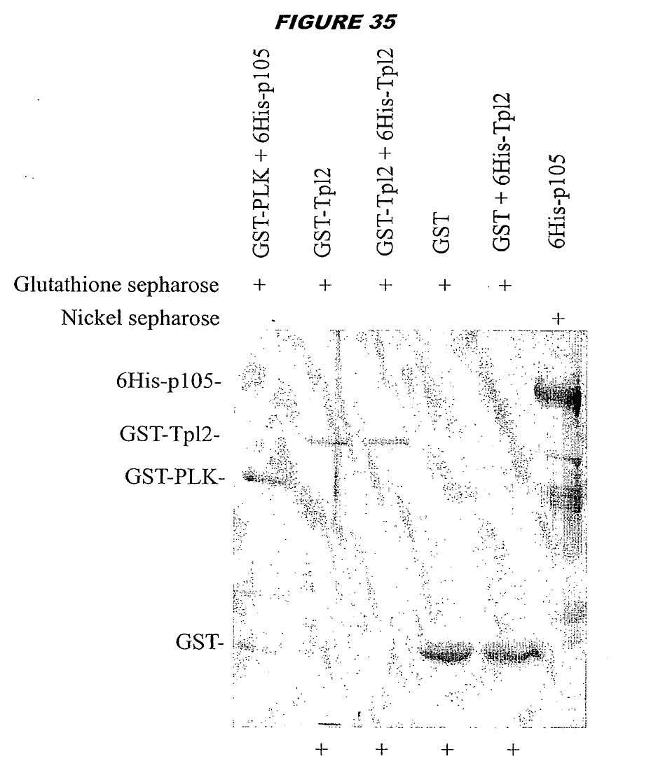

As Tpl2 and p105 interact in vivo, one test of the functionality of the proteins produced in yeast was to test for their interaction in vitro (FIG. 35). Glutathione sepharose beads loaded with GST-Tpl2, GST, or GST-PLKΔ (see FIG. 31) were mixed with 6His-p105 that had been eluted from a nickel sepharose column (see FIG. 33). Lane 3 of FIG. 35 shows that 6His-p105 was retained by the GST-TPL2 beads but not by beads carrying GST (lane 5) or GST-PLKΔ (lane 1). Thus p¹⁰⁵ and Tpl2 produced in this yeast system are able to interact in vitro as they do in vivo.

Expression of Other Proteins

Essentially similar procedures were used to produce GST-tagged S. cerevisiae Cdc16, Bfa1, Bub2, Tem1 and three deletion derivatives of C146 that contain the cyclin box domain. With Bfa1 and the Clb6 deletions, over-expression of the expressed proteins was toxic and reduced cell growth during the galactose induction period. To compensate for this, 500 ml of overnight culture of these cells in YEP+2% raffinose was used to inoculate a further 1 litre of YEP medium with a final concentration of 2% galactose. Induction then proceeded for 3-4 h before harvesting.

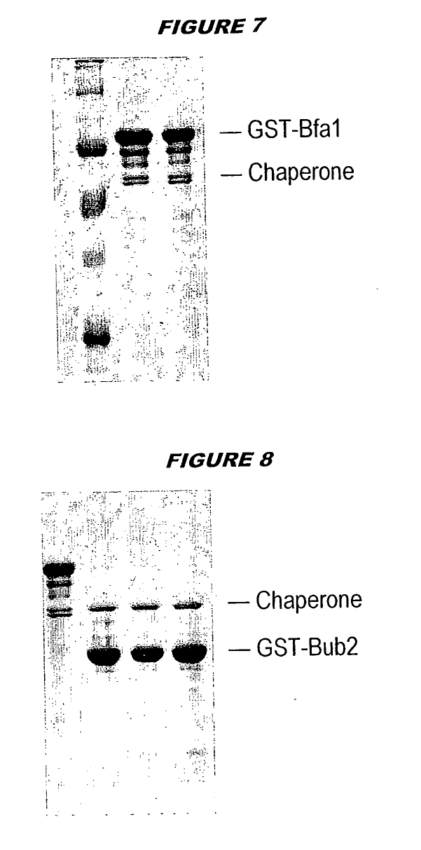

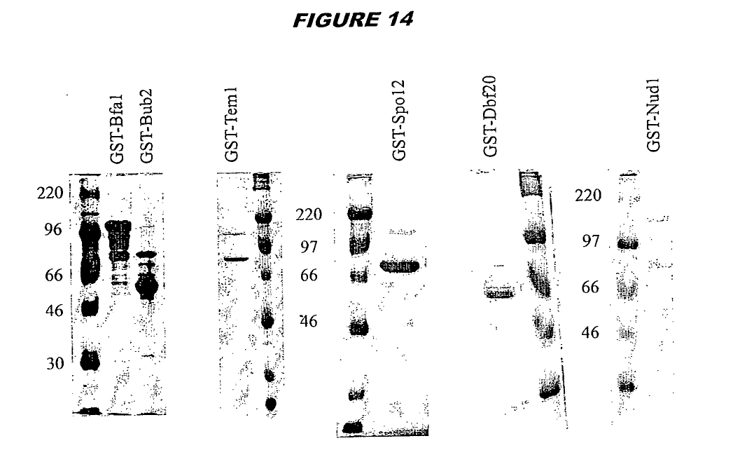

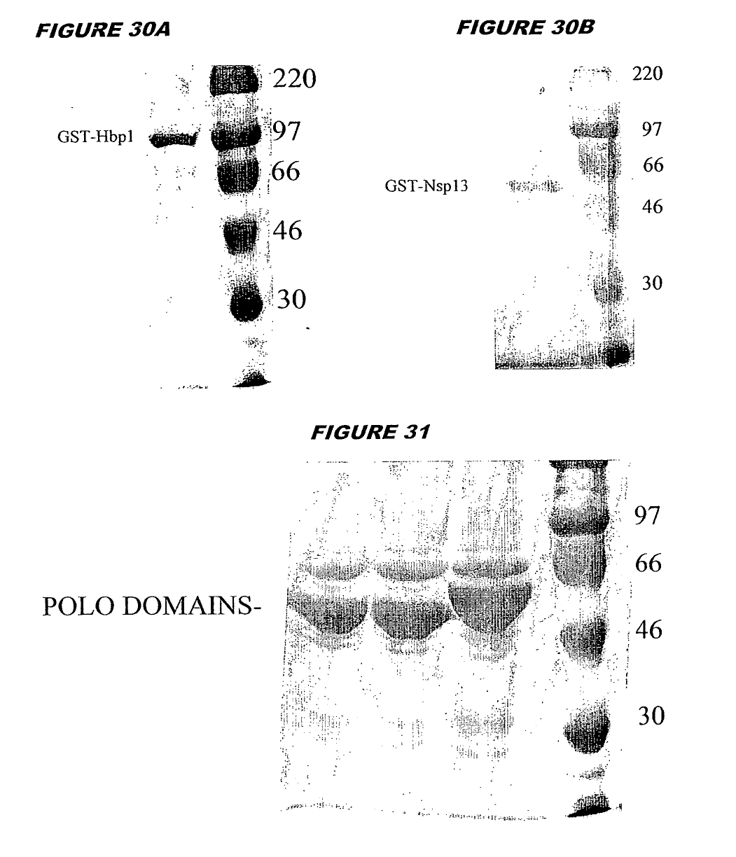

The MOB1 expression system of the invention has been used to express full size Bfa1 (FIG. 7), Bub2 (FIG. 8), Lte1 (FIG. 9), Tem1, Cla4, Net1, Nud1, Dbf20, Spo12 (FIG. 14), wild type and kinase-dead Cdc15, TPL2 (FIG. 10), an oncogenic C-terminally deleted TPL2, TPL2 deleted for 30 N-terminal residues, TPL2 deleted for both 30 N-terminal and 70 C-terminal residues, a kinase dead mutant of TPL2, the SARS virus Nsp13 putative mRNA cap-1 methyltransferase (e.g. FIG. 30 which shows 6His-tagged SARS virus Nsp13 methyl transferase) and three deletion derivatives of Clb6. All of these proteins have long histories of being difficult or impossible to produce in other systems but all of them give a GST-fusion product using the MOB1 system of the invention.

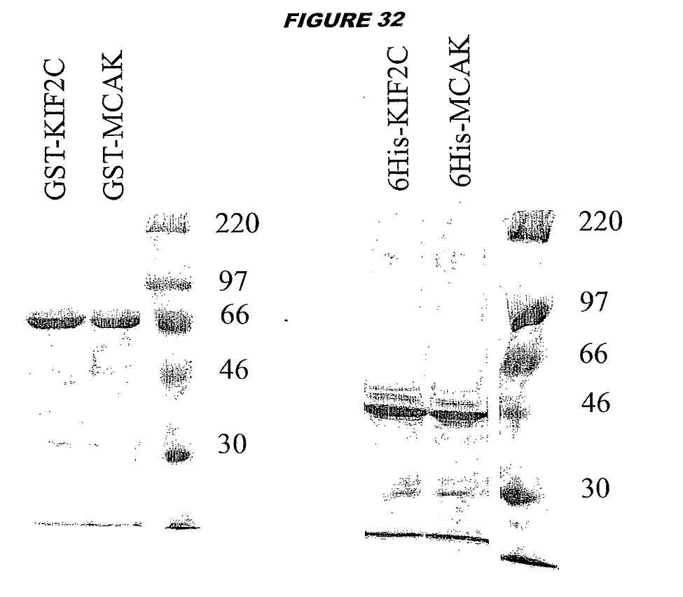

The following mammalian proteins have also been expressed in yeast using the method of the invention: GST-HBPI, a histone binding protein from mouse (FIG. 30); GST-fusions with fragments of the polo domain of human PLKI kinase (FIG. 31); 6His- and GST-tagged mouse kinesin KIF2C (FIG. 32); 6His- and GST-tagged CHO kinesin MCAK (FIG. 32); rat GST-TPL2, a kinase involved in the regulation of the immune and inflammatory responses (FIG. 33); human 6His-p105, a precursor of the NFKB transcription factor and regulator of TPL2 (FIG. 33); and human Abin2, a protein which interacts with Tpl2 (FIG. 33).

The Mitotic Exit Network

The Mitotic Exit Network (MLEN) of S. cerevisiae controls the final phase of mitotis. The activity of the MEN is governed by a small GTPase called Tem1 which in turn is negatively regulated by a two component GTPase activator protein (GAP) formed from the Bfa1 and Bub2 proteins. Positive regulation of Tem1 is thought to be provided by Lte1, a putative nucleotide exchange factor whose activity appears to be influenced by the kinase Cla4. Tem1 determines the activity of a kinase cascade comprising Cdc15 and Dbf2 and its cofactor, Mob1. Dbf20 is a homologue of Dbf2. Downstream effectors of Dbf2/Mob1 include the protein phosphatase Cdc14. Cdc14 is partly regulated by combining with Net1 in an inaccessible form in the nucleolus. Dbf2/Mob1 may also affect the activity of the protein degradation pathway specified by the ubiquitin ligase, APC complex Lte1 is a large yeast protein (>1400 amino acids). It could not be expressed as a GST-fusion protein using either the pGEX-KG E. coli expression system or the pBacPak baculovirus system. In contrast, expression using the MOB1 system of the invention gave high-level expression of the fusion protein in soluble form (FIG. 9).

Tem1 is a small Ras-like GTP-binding protein in the regulatory cascade of the mitotic exit network [34,59]. Expression in E. coli was attempted with a variety of vectors: pGEX-KG (GST-fusion) and pET28 (hexahistidine tag) did not give useful expression although small quantities of NIBP-Tem1 were obtained from a pMAL-c2X vector [34]. Expression of N-terminal fragments (amino acids 1-228 or 1-190) and of a Q79L mutant were also tested in various E. coli vectors, with no success. A hexahistidine fusion was tested without success in P. pastoris using the pPICZB vector, and the pBakPak8 GST-fusion system also failed in baculovirus. In contrast, expression using the MOB1 system of the invention gave high-level expression of the GST-Tem1 fusion protein in soluble form.

Bub2 is part of a GTPase-activating protein complex involved in the mitotic exit network [34]. Expression of Bub2 was attempted in E. coli using the vectors pGEX-KG, pMAL-c2X and pET28 but only the GST-fusion was expressed and this was with large amounts of E. coli GroEL chaperone protein. Expression of fragments (amino acids 36-258) and of a GST-Bub2-His₆ protein were also tested in various E. coli vectors, with no success. The pPICZαA vector failed in P. pastoris, as did the pBalPak8 and pBAC4X vectors in baculovirus. In contrast, expression using the MOB1 system of the invention gave high-level expression of the GST-Bub2 fusion protein in soluble form (FIG. 8).

Bfa1 is the other half of the GTPase-activating protein complex (Bfa1/Bub2) [34]. Expression of Bfa1 was attempted in E. coli using the vectors pGEX-KG, pGEX-His and pMAL-2c. Only MBP-fusion proteins could be expressed successfully. The pPICZB vector failed in P. pastoris, as did the pBakPak8 vector in baculovirus. In contrast, expression using the MOB1 system of the invention gave high-level expression of the GST-Bfa fusion protein in soluble form (FIG. 7). GST-Nsp13 expressed from pGEX-6P-2 in E. coli was insoluble but soluble GST-Nsp13 was obtained using the MOB1 system. After cleavage of the fusion protein with human rhinovirus protease (PreScission Protease) yields were approximately 1 mg Nsp13/litre of induced cells.

FIG. 14 shows glutathione sepharose affinity purification of GST-Tem1 and its negative regulators GST-Bfa1 and GST-Bub2. Bub2 has sequence homology with canonical GTPase activating proteins (GAPs) but is only active as a GAP when associated with Bfa1.

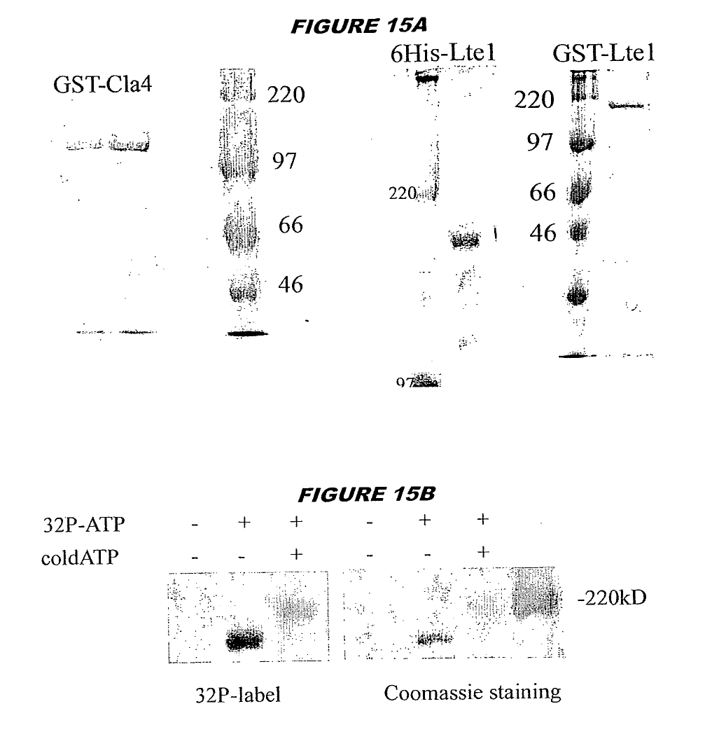

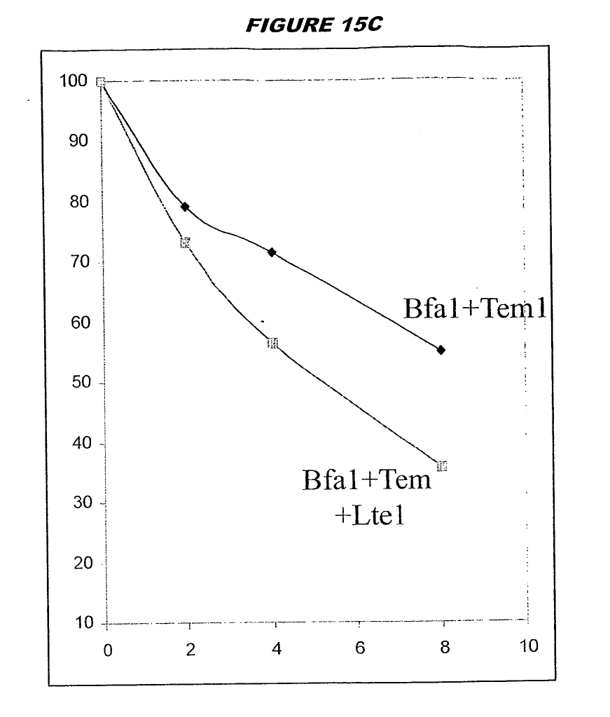

Lte1 has been expressed as either a GST- or 6His-fusion protein from either pMG1 or pMH919-Lte1's putative regulatory kinase Cla4 has also been expressed as a GST-fusion protein. These proteins have been purified by affinity chromatography (FIG. 15A). In in vitro kinase assays, Cla4 is able to phosphorylate 6His-Lte1, as judged by both the incorporation of radioactive label from γ³² P ATP and, with excess ATP, by the decrease in electrophoretic mobility typical of modified proteins (FIG. 15B).

The putative nucleotide exchange activity of Lte1 was confirmed in in vitro assays, which monitored the loss of radiolabelled GDP from the Tem1/Bfa1 complex. In this assay, addition of Lte1 accelerated the loss of GDP consistent with the activity of an exchange factor (FIG. 15C). Thus, the recombinant 6His-Lte1 produced in yeast displayed its predicted biochemical activity in vitro.

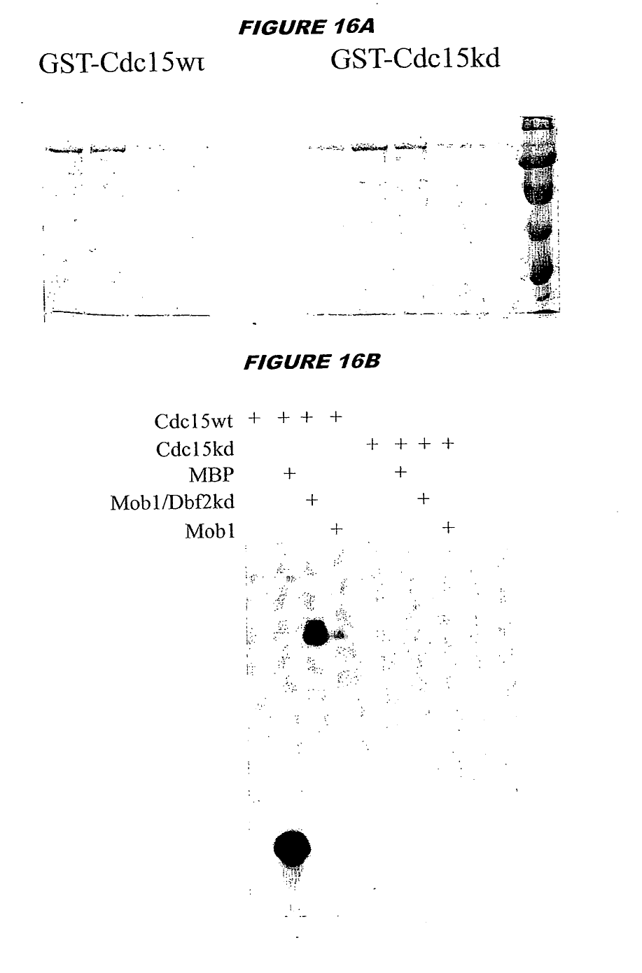

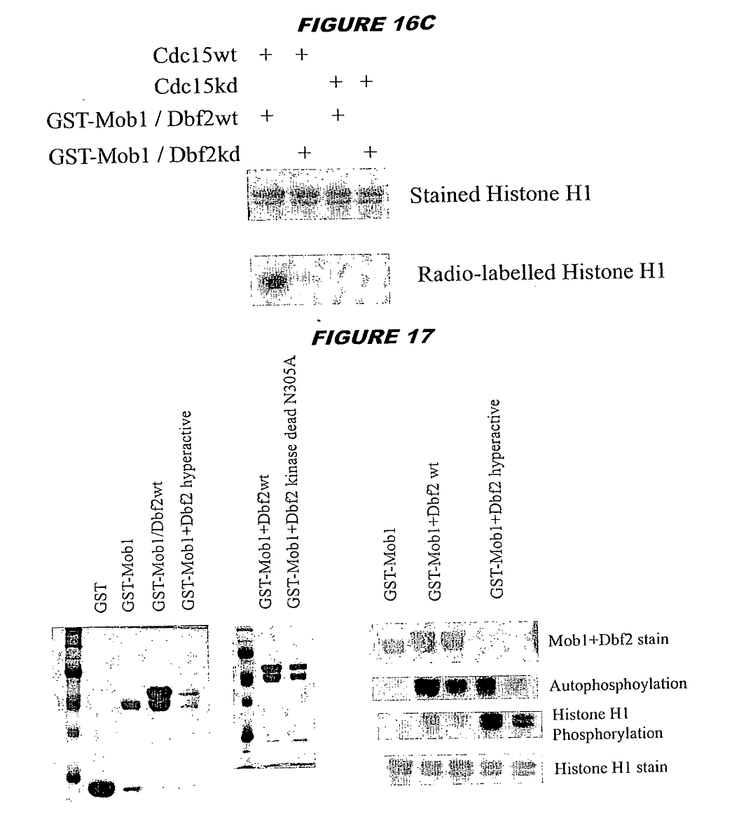

The kinase Cdc15 is the downstream effector of Tem1. Wild type and kinase dead (K54L) forms of GST-Cdc15 (FIG. 16A) have been produced using the expression system of the invention. FIG. 16B shows that wild type GST-Cdc15 phosphorylated OST-Mob1, GST-Mob1, GST-Mob1+Dbf2 N305A, and the artificial substrate, myelin basic protein. The kinase dead form of GST-Mob1+Dbf2N305A was used as a substrate here to eliminate additional phosphorylation events produced by this second kinase. GST-Cdc15 with a K54L mutation in the kinase site was unable to phosphorylate any of these substrates. Thus, Cdc15 can be prepared using the expression system and displays the biochemically appropriate activities in vitro.

The GST-Mob1/Dbf2 kinase dead complex mentioned above was produced by a variant of plasmid pMG1 which was reconfigured to express GST-MOB1 from the GAL1-10 promoter rather than the native MOB1 (FIG. 11B) This was possible because GST-MOB1 is still able to complement and maintain the viability of a Δmob1 strain. Untagged Dbf2 was expressed from the other side of the GAL1-10 promoter (FIG. 11B). Because of the stoichometric binding of Dbf2 with Mob1 it was possible to prepare untagged Dbf2 by co-purification with GST-Mob1. Wild type (wt), N305A kinase dead (kd), and hyperactive forms of Dbf2 were prepared in this way (FIG. 17).

The kinase activity of GST-Mob1+wild type and mutant forms of Dbf2 was examined. Both wild type and hyperactive kinases were able to phosphorylate the artificial substrate, Histone H1 (FIG. 17C), although phosphorylation was more efficient with the hyperactivated form of Dbf2. In addition, wild type and hyperactive GST-Mob1+Dbf2 displayed autophosphorylation (FIG. 17C) while the kinase dead form did not (FIG. 19).

Furthermore, when GST-Mob1+wild type Dbf2 was phosphorylated by Cdc15, then Dbf2 kinase activity towards Histone H1 was increased (FIG. 16C). This is in agreement with earlier data obtained by different means and so indicates that properly functional Mob1+Dbf2 complex is produced by the yeast expression system of the invention.

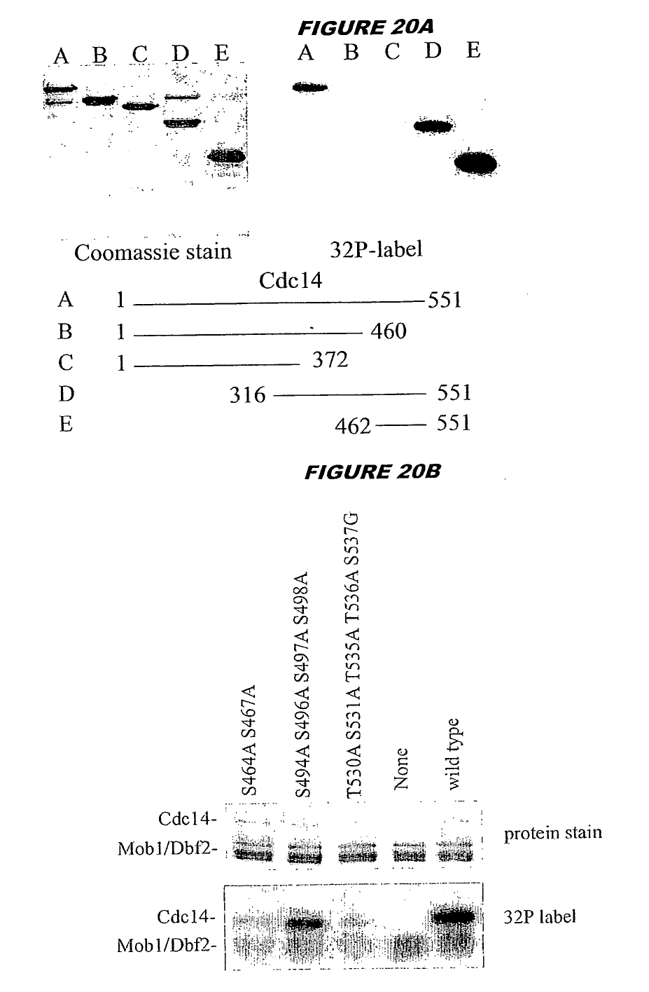

The natural substrates of Mob1+Dbf2 kinase have not previously been reported. However these results show that this kinase has activity in vitro towards components of the APC ubiquitin ligase complex (FIG. 19 ) and to the downstream MEN effector, Cdc14 (FIG. 20).

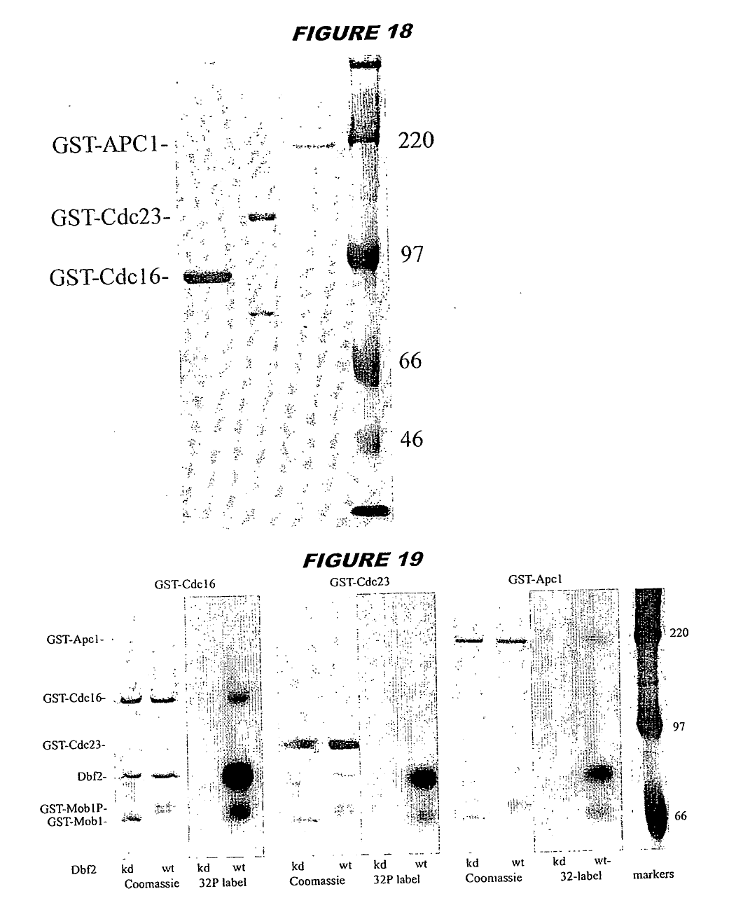

GST-Apc1, GST-Cdc16 and GST-23 were individually prepared using the yeast expression system (FIG. 18). GST-Apc1 and GST-Cdc16 were both phosphorylated by GST-Mob1+wild type Dbf2 but GST-Cdc23 was not (FIG. 19). Autophosphorylation of GST-Mob1+wildtype Dbf2 was also clearly seen. In contrast, control GST-Mob1+kinase dead Dbf2 was unable to phosphorylate any of these substrates or undergo autophosphorylation.

The above data therefore show that a complex of GST-Mob1 with wild type and mutant forms of Dbf2 kinase can be purified using the yeast expression system of the invention and that these complexes display the appropriate biochemical activities in vitro.

Cdc 14 is known to be a phosphatase and effector of several events at the end of mitotic exit. GST-Cdc14 was produced in the yeast expression system and proved to be a good substrate for GST-Mob1 kinase activity (FIG. 20). Deletion and point mutant forms of GST-Cdc14 were produced to map the sites of in vitro phosphorylation by GST-Mob1+Dbf2. By using four deletion derivatives phosphorylation was mapped to the C-terminal region of Cdc14 (FIG. 20). Point mutations at several putative phosphorylation sites in these region of the purified GST-Cdc14 further localised the amino acids subject to Mob1/Dbf2 kinase activity (FIG. 20B).

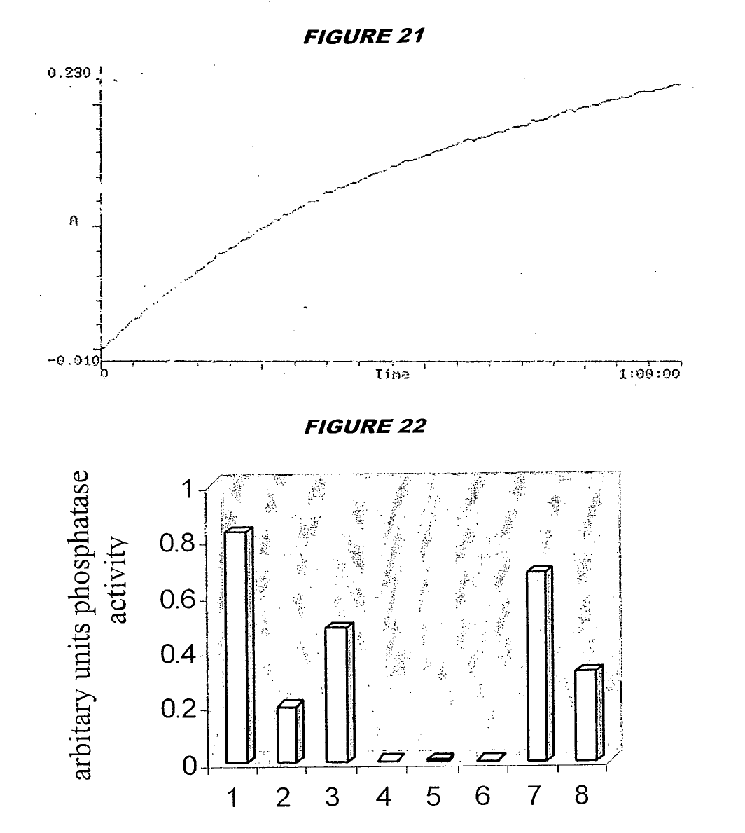

The functionality of these forms of Cdc14 was assayed in vitro by using the chromogenic phosphatase substrate, p-nitrophenyl phosphate. Phosphatase activity on p-nitrophenyl phosphate can be detected spectrophotometrically by an increase in absorbance at 410 nm. FIG. 21 shows the phosphatase activity of full length, wild type GST-Cdc14. The relative in vitro phosphatase activity of wild type GST-Cdc14 and several multiple point mutant derivatives are presented in FIG. 22.

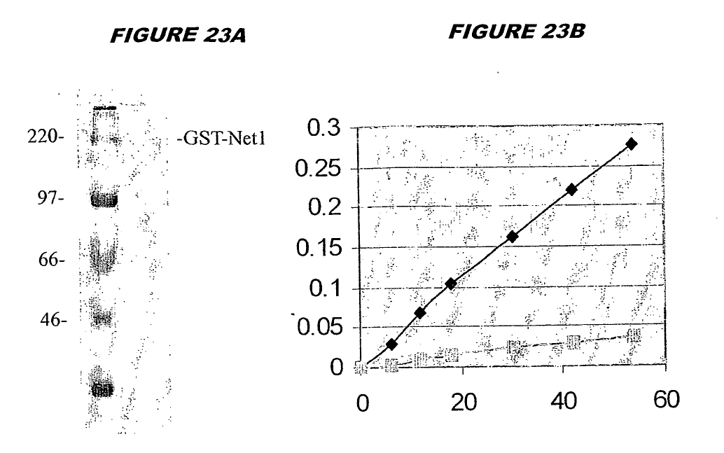

Finally, Cdc14 activity in vivo is blocked by interaction with the nucleolar protein Net1. GST-Net1was produced using the expression system (FIG. 23A) and tested for its effects on Cdc14 activity. The addition of GST-Net1 clearly reduced the in vitro phosphatase activity of GST-Cdc14 (FIG. 13). Thus, GST-Cdc14 produced with the yeast expression system has the appropriate phosphatase activity in vitro and, as in vivo, it can be negatively regulated GST-Net1.

Further Yeast Proteins

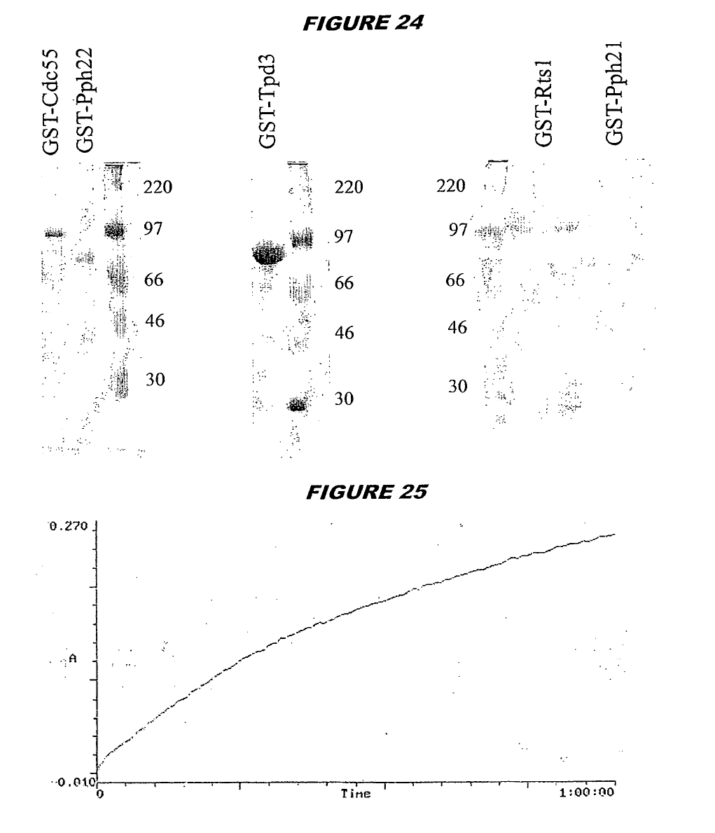

PP2A (S. cerevisiae Protein phosphatase 2A) is a multifunctional protein phosphatase. In budding yeast the Tpd1 subunit acts as a scaffold to two alternative enzymatic subunits, Pph21 or Pph22, and one of two alternative regulatory subunits, Cdc55 or Rts1. All five subunits can be expressed as GST-fusion proteins in the yeast expression system of the invention (FIG. 24). When GST-Cdc55 was prepared from yeast it was active as judged by its ability to use p-nitrophenyl phosphate as a substrate (see above). The raw data for this activity showing an increase in absorbance of the in vitro reaction mixture at 410 nm are presented in FIG. 25. In the preparation of GST-Cdc55 sufficient amounts of endogenous PP2A components were co-purified to permit activity.

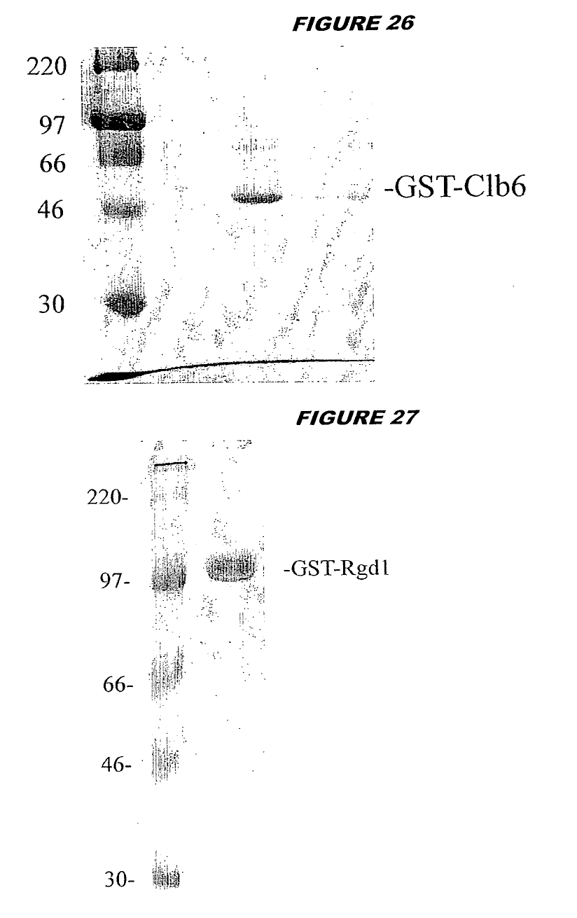

Clb6 (S. cerevisiae) is one of nine cyclin regulators of Cdc28, the major budding yeast cell cycle regulatory kinase. Three deletion derivatives of Clb6 expressing the so-called cyclin box were expressed as GST-fusion proteins (FIG. 26).

Rgd1 (S. cerevisiae) is a GTPase activating protein for the GTPase Rho. GST-Rgd1 was expressed from plasmid pMG1 in the MGY70 expression strain (FIG. 27).

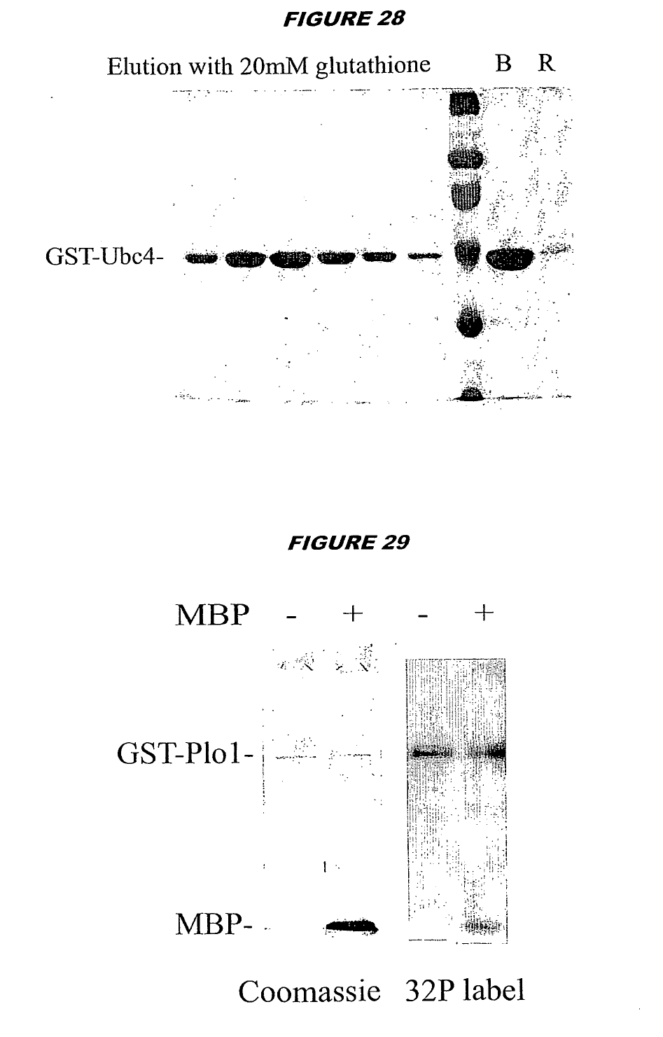

Ubc4 (S. cerevisiae) is an E2 ubiquitin conjugating enzyme which acts with the APC complex to ubiquitinate proteins and so direct them for protein degradation. A large scale preparation of GST-Ubc4 was undertaken to quantitate the yield of expressed protein. FIG. 28 shows the GST-Ubc4 eluted with reduced glutathione from a glutathione-sepharose column. It also shows that less than 5% of material was retained by the purification matrix after elution. 5 mg GST-Ubc4 was prepared from 25 g of induced cells.

Plo1 (Schizosaccharomyces pombe) is a multifunctional regulatory kinase that acts in the cell cycle. Plo1 is a member of the Polo group of kinases. Plo 1 was expressed in S. cerevisiae MGY70 as a GST-fusion protein and displayed in vitro kinase activity towards myelin basic protein (MBP) (FIG. 29).

Optimisation of Expression—Galactose Requirement for Inductions of Expression

Expression of recombinant genes usign pMG1 is induced by growth in rich medium with galactose as carbon source. In routine yeast culture carbon sources arc arbitrarily provided at 2%. In larger scale preparations considerable amounts of galactose might be used. Therefore, the minimum level of galactose actually required for induction was determined. Also, as the costs of this ingredient can vary by approximately five fold, cultures were tested whether there was any appreciable difference between the cheapest and most expensive forms of galactose.

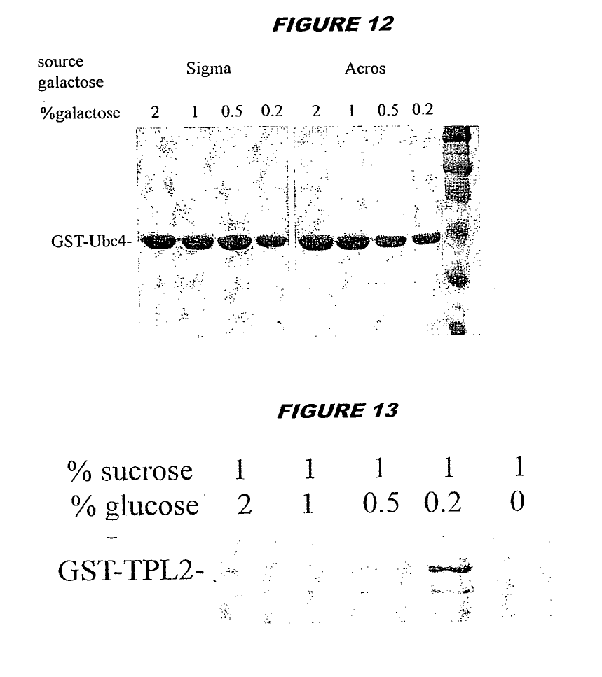

An expression strain was constructed from the standard expression host MGY70 containing a derivative of pMG1 expressing S. cerevisiae Ubc4 as a GST-fusion protein. FIG. 12 compares the yields of GST-Ubc4 when expression was induced with 2%, 1%, 0.5% or 0.2% galactose. The experiment also compared the efficacy of galactose from two manufacturers differing in price by 6-fold. The results show that 1% galactose from either source is sufficient for induction. Although yields with 0.5% of the more expensive galactose are slightly higher than with the cheaper galactose, it less expensive to use 1% of the cheaper galactose as the routine means of inducing expression. Thus while the more expensive galactose may be more appropriate for pharmaceutical preparation to ensure the highest levels of purity are maintained in accordence with good manufacturing practice, the cheaper galactose may be used in experimental conditions with no detrimental effects to the results obtained.

Optimisation of expression—Use of glucose prior to induction.

The expression system can include a mechanism by which copy number of the expression plasmid is increased to compensate for the effect of glucose in reducing the expression of the MOB1 selection gene from the GAL1-10 promoter. This mechanism was demonstrated in two ways.

First, glucose was shown to increase the plasmid copy number when the selection gene is expressed from GAL1-10 promoter. The copy number of two plasmids of comparable sizes was assessed where expression of the selective MOB1 gene was controlled either by the GAL10 promoter or by the natural MOB1 promoter. 10⁸ yeast cells carrying one plasmid or the other were grown in rich medium containing 1% glucose. Relative plasmid numbers were quantified by extracting DNA and performing transformations of competent E. coli DH5 with equal volumes of plasmid preparations from the two types of yeast.

| Plasmid MOB1 gene expressed from | Yield E. coli transformants |
|---|---|
| MOB1 promoter | 565 |
| GAL10 promoter | 1105 |

The table shows that when MOB1 is expressed from the GAL1-10 promoter there is an approximately two fold increase in plasmid copy number. This is the result expected if glucose repression of the GAL1-10 promoter limited the supply of the expression of essential Mob1 protein and forced a compensatory increase in copy number.

A second assay directly determined the effect of glucose expression of a cloned gene carried by pMG1. An expression strain was constructed from the standard expression host MGY70 containing a derivative of pMG1 expressing mouse TPL2 as a GST-fusion protein. Prior to induction of expression by growth in medium containing 1% galactose, overnight ‘precultures’ were grown in 1% sucrose plus glucose at 1%, 0.5%, 0.2%, 0.05% or 0%. After 6 h induction in 1% galactose medium. GST-TPL2 was prepared (FIG. 13). The yield of GST-TPL2 was greatest when 0.05% glucose was included in the preculture. Greater amounts of glucose were less effective, possibly because residual amounts might remain in the induction culture and antagonise the subsequent galactose induced activation of the GAL1-10 promoter. Therefore the invention only requires very low levels of glucose for induction of expression, thus reducing costs.

Hetero-Oligomers

Although MOB1 has been used as the selection essential gene for all the wort described above this section shows that, by employing a second essential gene for selection, a yeast expression system has been constructed to express two recombinant proteins simultaneously from two expression plasmids.

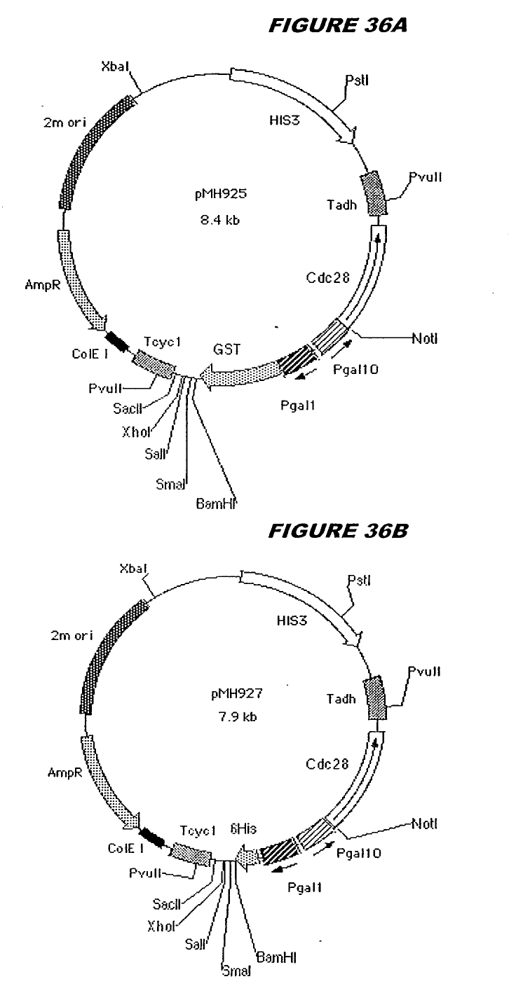

One class of expression plasmid includes all the MOB/TRP1-based vectors described above and in FIGS. 3 and 11. The second class of expression plasmids utilise the essential gene CDC28 for selection, rather than MOB1, and have HIS3 as an auxotrophic marker instead of TRP1. pMH925 is designed to produce proteins with a GST tag and pMH927 is designed to make 6His-tagged products (FIGS. 36A&B). The two classes of plasmids both use the divergent GAL1-10 promoter and can express either GST- or 6His-fusion proteins. The expression cells have chromosomal deletions of essential MOB1 and CDC28 genes which are made by the methods described above. They are kept alive by a third, covering plasmid which has a URA3 selective marker and which expresses both MOB1 and CDC28 genes from their endogenous promoters.

Use of this system is essentially the same as the single expression system. Coding sequences are cloned into the two types of expression vectors. The vectors are transformed into the expression strain selecting for trytophan and histidine prototrophy. The transformants are grown on medium containing 5-fluoro-orotic acid to select for loss of the ‘covering’ URA3 MOB1 CDC28 plasmid. The loss of the covering plasmid produces a strain carrying two different expression plasmids whose presence is maintained by selection for their essential MOB1 and CDC28 genes.

An example of the use of this system is shown where two proteins are co-expressed and, because of their known affinity for each other, they also co-purify (FIG. 37). A pMH925, CDC28-based plasmid encoding GST-TPL2 was co-expressed with either a pMH919 derivative expressing 6His-p105 or the ‘empty’ pMH919 vector expressing only the 6His affinity tag. Additional control cells expressed the GST affinity tag from pMH925 with a pMH919 derivative expressing 6His-p105. Lysates were prepared from these cells and GST- and 6His-tagged proteins were recovered by affinity purification with both glutathione sepharose and nickel sepharose. This experiment shows that GST-Tpl2 can be expressed from plasmids relying on a second essential gene, CDC28, for self selection (lane 1). GST is also expressed from the CDC28-based vector which was co-expressed with 6His-p105 (lane 3). As expected, the 6His-p105 that was co expressed with GST was not recovered using glutathione sepharose in lane 3, but it was seen using nickel sepharose purification (lane 6). Thus two different proteins can be co-expressed.

Co-expression was also seen in extracts from cells encoding GST-Tpl2 and 6His-p105. GST-Tpl2 was recovered after purification with glutathione sepharose (lane 2) while 6His-p105 was purified from the same cells with nickel sepharose. Importantly, 6His-p105 also co-purified with the GST-Tpl2 on glutathione sepharose (lane 2) but not with GST alone (lane 3). This indicates specific co-purification of 6His-p105 with GST-Tpl2. Similarly, GST-Tpl2 co-purified with 6His-p105 on nickel sepharose (lane 5) but not with the 6His tag alone (lane 4). Thus the GST-Tpl2 and 6His-p105 are co-expressed in forms that are able to interact and so co-purify.

In further examples, yeasts are made with chromosomal deletions of both MOB1 and CDC33. To complement the deletions, yeast are kept alive by a ‘covering’ plasmid expressing both MOB1 and CDC33 and carrying a URA3 selective marker. To insert the heterologous gene products, one plasmid is pMG1 as described above and the other is a similar plasmid where (a) MOB1 is replaced by CDC33 and (b) conditional selective marker HIS3 replaces TRP1. To allow separate purification, the second plasmid uses an epitope tag, a hexahistidinyl tag or no tag rather than a GST fusion.

Heterologous sequences are cloned into the two expression plasmids. The two plasmids are co-transformed into a yeast host, selecting for Trp⁺ and His⁺ prototrophy. Cells that have lost the URA3-covering plasmid are selected on FOA to give a cell capable of expressing two different proteins.

In related work, GST-Mob1 was expressed with untagged Dbf2 in mob1-deleted cells. Dbf2 is a kinase and Mob1 is an accessory protein required for activity. The divergent GAL1-10 promoter expressed GST-Mob1 in one direction and untagged Dbf2 in the other. Purification of GST-Mob1 on glutathione sepharose also yielded approximately equimolar amounts of untagged Dbf2, demonstrating how hetero-oligomers can be purified.

Expression in Escherichia coli

An E. coli BL21 derivative with good induction and protein stability characteristics is selected.

An essential gene for chromosomal deletion is chosen.

A covering plasmid based on pACYC184 is prepared, including: (a) the essential gene, prepared by PCR from E. coli genomic DNA and including its natural promoter and regulatory sequences; (b) the conditionally-lethal sacB marker to allow counter-selection during confirmation of chromosomal deletion and during plasmid shuffling; (c) a P15A replication origin; (d) a chloramphenicol selection marker. The plasmid is transformed into E. coli in preparation for deletion of the essential chromosomal gene.

After introduction of the covering plasmid, the chromosomal copy of the essential gene is replaced with a drug resistance marker using the methods described in reference 47 or 48. The drug resistance marker allows inheritance of the modified gene to be followed. Confirmation that the essential gene is provided by the covering plasmid and not by the chromosome can be provided by attempting to grow a bacterium in sucrose-based medium.

An expression plasmid based on pETDuet (Novagen™ ) is prepared, including: (a) the essential gene; (b) a mammalian, viral or other eukaryotic gene of interest; (c) two multiple cloning sites adjacent to tandem T7lac inducible promoters, with one MCS including a hexa-His tag; (d) a colE1 replication origin, which is compatible with the P15A origin used in the covering plasmid; and (e) an ampR gene, which allows the plasmid to be distinguished from the covering plasmid. The two genes (a) and (b) are under the control of the two T7lac promoters. A simpler system uses a normal pET or pGEX vector, with only a single MCS for receiving the mammalian gene; the essential gene with its own promoter is first cloned into a non-MCS site.

The expression plasmid is transformed into the E. coli to give a bacterium carrying both the covering plasmid and the expression plasmid.

Loss of the covering plasmid is then selected by growing bacteria on sucrose. This growth stage can be preceded by a period of growth in the absence of chloramphenicol, in order to provide an opportunity for ‘natural’ loss of the covering plasmid. After the sucrose counterselection, loss of the covering plasmid is confirmed by checking for chloramphenicol sensitivity. After this confirmation there is no need for further use of antibiotics during growth as the expression plasmid can be maintained by its providing the essential gene rather than by its ampR gene. The bacteria can thus be grown through several cultures in order to eliminate any trace of chloramphenicol, thereby giving an antibiotic-free preparation of bacteria which can be used to express the mammalian protein without antibiotic contamination.

Bacteria are cultured and then induced under standard condition using IPTG. The mammalian protein is expressed as a GST fusion protein which is then purified using the appropriate affinity column. The native protein is released using thrombin cleavage to give a final purified product.

In a further development, the expression plasmid includes the oriV/TrfA replicon system for copy number amplification, as disclosed in reference [60].

It will be understood that the invention has been described by way of example only and modifications may be made whilst remaining within the scope and spirit of the invention.

### REFERENCES (THE CONTENTS OF WHICH ARE HEREBY INCORPORATED IN FULL)

[1] Cereghino & Cregg (2000) FEMS Microbiology Reviews 24:45-66.
[2] çakar et al. (1999) Biotechnol Letters 21:611-616.
[3] http://magpie.genonme wisc.edu/˜chris/essential.html
[4] http://www.shigen.nig.ac.jp/ecoli/pec/index.jsp
[5] http://www.mips. biochem.mpg.de/proj/yeast/tables/essential/lethal.html
[6] European Saccharomyces Cerevisiae Archive For Functional Analysis. See http:/www.rz.uni-frankfurt.de/FB/fb16/mikro/euroscarf/data/less.html
[7] Dujon (1998) Electrophoresis 19:617-624.
[8] http://mips.gsf.de/proj/eurofan/eurofan_{—}2/b3/
[9] Arigoni et al. (1998)Nature Biotechnol 16:851-856.
[10] Lamichhane et al. (2003) PNAS USA 100:7213-7218.
[11] Haselbeck et al. (2002) Curr Pharm Des. 8:1155-1172.
[12] http//tubic.tju.edu.cn/deg/
[13] Zhang et al. (2004) DEG, a Database of Essential Genes. Nucleic Acids Research, in press
[14] Sharp & Li (1987) Nucleic Acids Res 15:1281-1295.
[15] Luca & Winey (1998) Mol Biol Cell 9:29-46.
[16] Anthony el al. (2001) J Biol Chem 276:39645-39652.
[17] Hohfeld & Hartl (1994) J Cell Biol 126:305-315.
[18] Sikorski & Boeke (1991) Methods Enzymol 194:302-318.
[19] Wang et al. (1993) PNAS USA 90:7874-7878.
[20] Nathan & Lindquist (1995) Mol Cell Biol 15:3917-3925.
[21] Elledge & Davis (1988) Gene 70:303-312.
[22] Lisowsky et al. (2002) Mol Biol Cell 7:2245-55.
[23] Erikson et al. (2004) Nature Biotech 22(4):455-458.
[24] Fairhead et al. (1996) Yeast 12:1439-1457.
[25] Frangioni & Neel (1993) Anal Biochem 210:179-187.
[26] Gaberc-Porekar & Menart (2001) J Biochem Biophys Methods 49:335-360.
[27] Brizzard et al. (1994) Biotechniques 16:730-735.
[28] Chong et al. (1997) Gene 192:271-281.
[29] Southworth et al. (1999) Biotechniques 27:110-120.
[30] Aslandis et al. (1990) Nucleic Acids Res 18:6069-6074.
[31] Haun et al. (1992) Biotechniques 515-518.
[32] Stols et al. (2002) Prot Exp & Purif 25:8-15.
[33] Keng et al. (1994) Yeast 10:953-958.
[34] Geymonat et al. (2002) J Biol Chem 277:28439-28445.
[35] Erhart & Hollenberg (1983) J Bacteriol 156:625-635.
[36] Johnston & Davis (1984) Mol Cell Biol 4:1440-1448.
[37] Nakao et al. (1986) Mol Cell Biol 6:2613-2623.
[38] Fermiñán & Dominguez (1998) Appl Environ Microbiol 64:2403-2408.
[39] Maundrell (1993) Gene 123:127-130.
[40] Moreno et al. (2000) Yeast 16:861-872.
[41] Shang el al. (2002) PNAS USA 99:3734-3739.
[42] Labbe & Thiele (1999) Methods Enzymol 306:145-153.
[43] Shimizu-Sato et al. (2002) Nature Biotechnol 20:1041-1044.
[44] Huh et al. (2003) Nature 425:686-691.
[45] Ghaemmaghami et al. (2003) Nature 425:737-741.
[46] Giga-Hama & Kumagai (1999) Biotechnol Appl Biochm 30:235-244.
[47] Datsenko & Wanner (2000) PNAS USA 97:6640-6645.
[48] Link et al. (1997) J Bacterial 179:6228-6237.
[49] Gomez et al. (1998) Mol Microbiol 29.:617-628.
[50] Ammerer et al. (1986) Mol Cell Biol 6:2490-2499.
[51] Otterstedt et al. (2004) EMBO Rep. 5(5):532-537.
[52] Hamilton et al. (2003) Science 301:1244-1246.
[53] Choi et al. (2003) PNAS USA 100:5022-5027.
[54] Sikorski & Hieter (1989) Genetics 122:19′-27.
[55] Johnston et al. (1990) Mol Cell Biol 10:1358-1366.
[56] Berben et al. (1991) Yeast 7:475-477.
[57] Christianson et al. (1992) Gene 110:119-122.
[58] Guan & Dixon (1991) Anal Biochem 192:262-267.
[59] Shirayama et al. (1994) Mol Cell Biol 14:7476-7482.
[60] Wild et al. (2002) Genome Res 12:1434-1444.

1. A cell that expresses both chromosomal genes and extra-chromosomal genes, wherein (a) the expressed extra-chromosomal genes include a gene with an essential function, the expression of which is unconditionally required for survival of the cell, (b) the expressed chromosomal genes do not provide that essential function, and (c) the extra-chromosomal genes include a heterologous gene, the expression of which is controlled by a promoter that is functional in the cell and wherein the extra-chromosomal genes are located on an extra-chromosomal vector.

2. A cell according to claim 1, where (d) the expressed extra-chromosomal genes comprise at least one further gene with a different essential function from (a), and (e) the expressed chromosomal genes also do not provide that essential function.

3. A cell according to claim 1, comprising at least one further extra-chromosomal heterologous gene, the expression of which is controlled by a promoter that is functional in the cell.

4. A method for expressing a heterologous gene, comprising the step of growing the cell of claim 1 in a culture medium.

5. A method for purifying a protein, comprising the steps of (a) growing the cell of claim 1 in a culture medium, such that it expresses said protein; and (b) purifying the protein.

6. The method of claim 5, further comprising the step of (c) treating the protein with a protease to provide a cleavage product of interest.

7. A cell that expresses both chromosomal genes and extra-chromosomal genes, wherein (a) the expressed extra-chromosomal genes include a gene with an essential function, the expression of which is unconditionally required for survival of the cell, (b) the expressed chromosomal genes do not provide that essential function, and (c) the extra chromosomal genes include a conditionally-lethal gene, wherein the essential gene is MOBI, Cdc33 or Hsp10.

8. A cell that expresses chromosomal genes, a first set of extra-chromosomal genes and a second set of extra-chromosomal genes, wherein (a) the expressed first and second sets of extra-chromosomal genes both include a gene with the same essential function, the expression of which is unconditionally required for survival of the cell, (b) the expressed chromosomal genes do not provide that—essential function, (c) the first set of extra chromosomal genes includes a conditionally-lethal gene, and (d) the second set of extra-chromosomal genes includes both a conditionally-required gene and a heterologous gene.

9. A cell according to claim 8, wherein (e) the cell also expresses a third set of extra chromosomal genes comprising a gene with a different essential function to that of the gene found in both the first and second set of extra-chromosomal genes, the expression of which is required for survival of the cell, (f) a conditionally required gene and a heterologous gene.

10. An extra-chromosomal vector, comprising: (a) an essential gene whose expression is unconditionally required for survival of a cell of interest; (b) a conditionally-required gene to allow selection of host cells which include the extra-chromosomal vector; and (c) a gene encoding a heterologous protein of interest operably linked to a promoter that is functional in the cell of interest.

11. The vector of claim 10, wherein the vector is a plasmid.

12. The vector of claim 10, wherein the conditionally-required gene is a resistance gene.

13. The vector of claim 12, wherein the resistance gene is an antibiotic resistance gene, a drug resistance gene, or a herbicide resistance gene.

14. The vector of claim 10, wherein the conditionally-required gene complements an auxotrophic mutation in the host's chromosome.

15. An extra-chromosomal vector, comprising: (a) an essential gene whose expression is unconditionally required for survival of a cell of interest; (b) a conditionally-lethal gene to allow selective killing of host cells which include the extra-chromosomal vector, wherein the essential gene is MOBI, Cdc33 or Hsp10.

16. The vector of claim 10, comprising one or more of the following elements: (i) an origin of replication functional in a host cell of interest; (ii) a polylinker containing a plurality of restriction sites; (iii) a transcription termination sequence downstream of one or more of the promoters and their coding sequences in the vector.

17. The vector of claim 10, comprising one or more of (iii) an origin of replication functional in bacteria; and (iv) an antibiotic resistance marker suitable for selection of bacterial transformants.

18. A method for preparing a cell according to claim 1, comprising the steps of (a) obtaining a first cell that expresses chromosomal genes, a first set of extra-chromosomal genes and a second set of extra-chromosomal genes, wherein (1) the expressed first and second sets of extra-chromosomal genes both include a gene with the same essential function, the expression of which is unconditionally required for survival of the cell, (2) the expressed chromosomal genes do not provide that essential function, (3) the first set of extra-chromosomal genes includes a conditionally-lethal gene, and (4) the second set of extra-chromosomal genes includes both a conditionally-required gene and a heterologous gene; (b) selecting transformants which express the vector's conditionally-required gene(s); and (c) selecting transformants which lose the conditionally-lethal gene(s).

19. The cell, method or vector of any preceding claim, wherein the essential gene is a gene whose loss prevents cell division, prevents mitosis, prevents transcription, or prevents translation.

20. The cell, method or vector of any preceding claim, wherein the essential gene has a coding sequence of <3000 base pairs.

21. The cell, method or vector of any preceding claim, wherein the essential gene is not lethal when hyper-expressed.

22. The cell, method or vector of any preceding claim, wherein the essential gene is MOBI.

23. The cell, method or vector of any preceding claim, wherein the heterologous gene comprises a sequence from a higher eukaryote or a eukaryotic virus.

24. The cell, method or vector of claim 23, wherein the eukaryote is an animal.

25. The cell, method or vector of any preceding claim, wherein the heterologous gene encodes a fusion protein comprising a first sequence and a second sequence.

26. The cell, method or vector of claim 25, wherein the junction between the first sequence and second sequence includes a protease recognition sequence.

27. The cell, method or vector of claim 26, wherein the protease is thrombin, factor Xa protease, enterokinase, endopeptidase rTEV or human rhinovirus protease 3C.

28. The cell, method or vector of claim 25, wherein the junction between the first sequence and second sequence includes an intein.

29. The cell, method or vector of any preceding claim, wherein the heterologous gene comprises a sequence encoding glutathione-S-transferase, a poly-histidine tag, a calmodulin-binding peptide, a maltose-binding protein, a chitin-binding domain, or an immunoaffinity epitope.

30. The cell, method or vector of any preceding claim, wherein the heterologous gene encodes a protein which forms oligomers.

31. The cell, method or vector of any preceding claim, wherein the heterologous gene is expressed as a soluble protein.

32. The cell, method or vector of any preceding claim, wherein expression of the essential gene is controlled by an inducible promoter.

33. The cell, method or vector of any preceding claim, wherein expression of the heterologous gene is controlled by an inducible promoter.

34. The cell, method or vector of claim 32, wherein the promoter is a repressible promoter.

35. The cell, method or vector of claim 34, wherein the heterologous gene and the essential gene are inducible and/or repressible by the same stimulus.

36. The cell, method or vector of any preceding claim, wherein expression of the essential gene and/or the heterologous gene is controlled by a galactokinase/UDP-glucose 4 epimerase promoter.

37. The cell, method or vector of any preceding claim, wherein the cell is a eukaryote.

38. The cell, method or vector of claim 37, wherein the eukaryote is a yeast.

39. The cell, method or vector of claim 38, wherein the yeast is Saccharomyces cerevisiae or Schizosaccharomyces pombe.

40. The cell, method of vector of any preceding claim, wherein the heterologous gene encodes a LteI protein, a BfaI protein, a Bub2 protein, a CDCS protein, a CDC15 protein, a CDC28 protein, a Tp12 protein, a SARS virus Nsp13 protein, or a mRNA Cap 1 methyl transferase protein.
